(19) United States
(12) Patent Application Publication
(10) Pub. No.: US 2025/0176799 A1
(43) Pub. Date: Jun. 5, 2025

(54) USER INTERFACES FOR NAVIGATING ANATOMICAL CHANNELS IN MEDICAL PROCEDURES

(71) Applicant: Auris Health, Inc., Santa Clara, CA (US)

(72) Inventors: Daniel Lau, Oakland, CA (US); Jialu Li, San Jose, CA (US); Jo Ann Sison, Redwood City, CA (US)

(73) Assignee: Auris Health, Inc., Santa Clara, CA (US)

(21) Appl. No.: 19/047,249

(22) Filed: Feb. 6, 2025

Related U.S. Application Data
(63) Continuation of application No. PCT/IB2023/058156, filed on Aug. 11, 2023.
(60) Provisional application No. 63/397,252, filed on Aug. 11, 2022.

(51) Int. Cl. A61B 1/00; A61B 1/05; A61B 34/00; A61B 34/20; A61B 34/30; A61B 34/37

(52) U.S. Cl. CPC A61B 1/0005; A61B 1/00006; A61B 1/00009; A61B 1/00149; A61B 1/05; A61B 34/20; A61B 34/25; A61B 34/37; A61B 2034/303

(57) ABSTRACT

A medical system includes a scope configured to capture at least one endoscopic image, a display device configured to display the endoscopic image, one or more processors, and a memory storing instructions for execution by the one or more processors, the stored instructions including instructions that cause the one or more processors to determine an orientation of the scope associated with the endoscopic image. The stored instructions may include instructions to determine at least one reference axis associated with a luminal network. The reference axis may indicate a first anatomical direction and a second anatomical direction. The stored instructions may include instructions to generate an orientation indicator based on the reference axis. The stored instructions may include instructions to cause the display to present the endoscopic image and the orientation indicator, the orientation indicator overlaid on the endoscopic image.

### RELATED APPLICATION(S)

This application is a continuation of International Application No. PCT/IB2023/058156, filed Aug. 11, 2023, and entitled “USER INTERFACES FOR NAVIGATING ANATOMICAL CHANNELS IN MEDICAL PROCEDURES,” which claims the benefit of priority to U.S. Provisional Application No. 63/397,252, filed Aug. 11, 2022, and entitled “USER INTERFACES FOR NAVIGATING ANATOMICAL CHANNELS IN MEDICAL PROCEDURES,” which are assigned to the assignee hereof. The disclosure of the prior applications are considered part of and are incorporated by reference in this patent application.

### BACKGROUND

### Field

The systems and methods disclosed herein are related to navigating a medical instrument through a patient's anatomy, and more particularly to a user interface which assists a user in navigating the medical instrument.

### Description of Related Art

Certain robotic medical procedures can involve the use of shaft-type instruments, such as endoscopes, which may be inserted into a patient through an orifice (e.g., a natural orifice) and advanced to a target anatomical site. Such medical instruments can be articulatable, such that the tip and/or other portion(s) of the shaft can deflect in one or more dimensions using robotic controls. During navigation of the medical instruments, various graphical elements may be provided on a display to assist operators in advancing the medical instruments to the desired target location.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an embodiment of a cart-based robotic system arranged for diagnostic and/or therapeutic bronchoscopy procedure(s).

FIG. 2 depicts further aspects of the robotic system of FIG. 1.

FIG. 3 illustrates an embodiment of the robotic system of FIG. 1 arranged for ureteroscopy.

FIG. 4 illustrates an embodiment of the robotic system of FIG. 1 arranged for a vascular procedure.

FIG. 5 illustrates an embodiment of a table-based robotic system arranged for a bronchoscopy procedure.

FIG. 6 provides an alternative view of the robotic system of FIG. 5.

FIG. 7 illustrates an example system configured to stow robotic arm(s).

FIG. 8 illustrates an embodiment of a table-based robotic system configured for a ureteroscopy procedure.

FIG. 9 illustrates an embodiment of a table-based robotic system configured for a laparoscopic procedure.

FIG. 10 illustrates an embodiment of the table-based robotic system of FIGS. 5-9 with pitch or tilt adjustment.

FIG. 11 provides a detailed illustration of the interface between the table and the column of the table-based robotic system of FIGS. 5-10.

FIG. 12 illustrates an alternative embodiment of a table-based robotic system.

FIG. 13 illustrates an end view of the table-based robotic system of FIG. 12.

FIG. 14 illustrates an end view of a table-based robotic system with robotic arms attached thereto.

FIG. 15 illustrates an exemplary instrument driver.

FIG. 16 illustrates an exemplary medical instrument with a paired instrument driver.

FIG. 17 illustrates an alternative design for an instrument driver and instrument where the axes of the drive units are parallel to the axis of the elongated shaft of the instrument.

FIG. 18 illustrates an instrument having an instrument-based insertion architecture.

FIG. 19 illustrates an exemplary controller.

FIG. 20 depicts a block diagram illustrating a localization system that estimates a location of one or more elements of the robotic systems of FIGS. 1-10, such as the location of the instrument of FIGS. 16-18, in accordance to an example embodiment.

FIG. 21 illustrates an example of a graphical user interface which provides either or both of a restricted field-of-view and an expanded field-of-view of an intraoperative image captured by an endoscope.

FIG. 22 illustrates an example of a graphical user interface which provides various views.

FIG. 23 illustrates an example of a graphical user interface including a plurality of orientation indicators.

FIG. 24 illustrates an example of a graphical user interface including an example of a two-dimensional (2D) compass.

FIG. 25 illustrates an example of a graphical user interface including an example of a three-dimensional (3D) compass.

FIG. 26 illustrates example articulation scenarios in association with articulation indicators.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts features associated with separate figures that are identified by common reference numbers are not related and/or similar with respect to at least certain aspects.

The present disclosure provide systems, devices, and methods for navigation of an instrument shaft, such as a medical endoscope, within a luminal network and monitoring of instrument shaft articulation. Articulation of instruments in accordance with the present disclosure can be implemented by tensioning one or more tendons, referred to herein as “pull wires,” that traverse a shaft of the instrument. With respect to medical instruments described in the present disclosure, the term “instrument” is used according to its broad and ordinary meaning and may refer to any type of tool, device, assembly, system, subsystem, apparatus, component, or the like. In some contexts herein, the term “device” may be used substantially interchangeably with the term “instrument.” Furthermore, the term “shaft” is used herein according to its broad and ordinary meaning and may refer to any type of elongate cylinder, tube, scope (e.g., endoscope), prism (e.g., rectangular, oval, elliptical, or oblong prism), wire, or similar, regardless of cross-sectional shape. It should be understood that any reference herein to a “shaft” or “instrument shaft” can be understood to possibly refer to an endoscope.

### 1. Overview

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

### A. Robotic System—Cart.

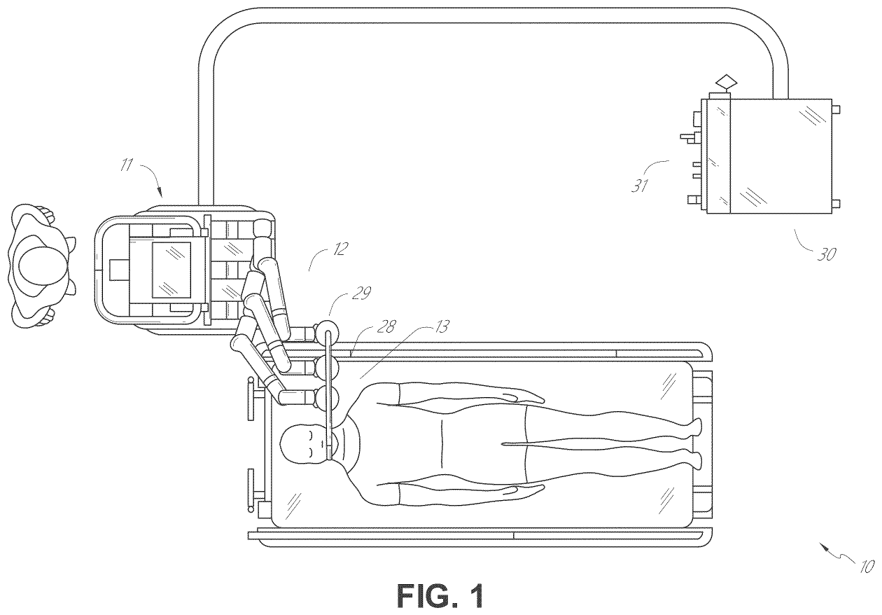

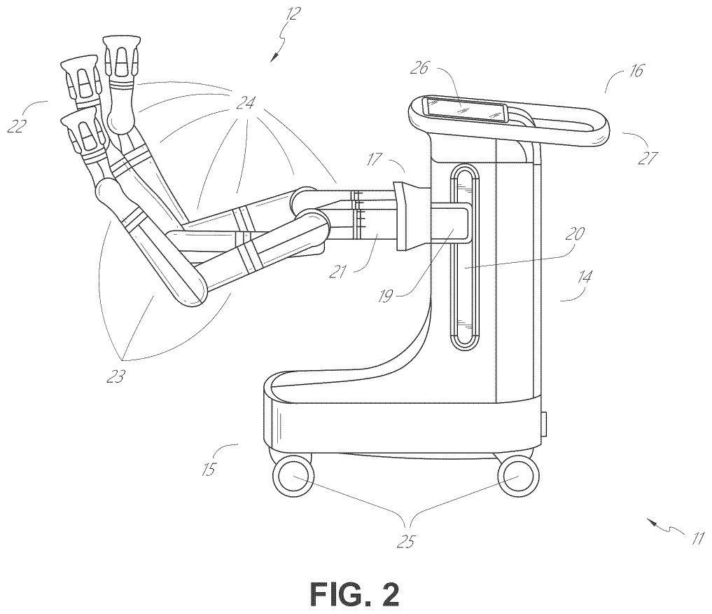

The robotically-enabled medical system may be configured in a variety of ways depending on the particular procedure. FIG. 1 illustrates an embodiment of a cart-based robotically-enabled system 10 arranged for a diagnostic and/or therapeutic bronchoscopy procedure. During a bronchoscopy, the system 10 may comprise a cart 11 having one or more robotic arms 12 to deliver a medical instrument, such as a steerable endoscope 13, which may be a procedure-specific bronchoscope for bronchoscopy, to a natural orifice access point (i.e., the mouth of the patient positioned on a table in the present example) to deliver diagnostic and/or therapeutic tools. As shown, the cart 11 may be positioned proximate to the patient's upper torso in order to provide access to the access point. Similarly, the robotic arms 12 may be actuated to position the bronchoscope relative to the access point. The arrangement in FIG. 1 may also be utilized when performing a gastro-intestinal (GI) procedure with a gastroscope, a specialized endoscope for GI procedures. FIG. 2 depicts an example embodiment of the cart in greater detail.

With continued reference to FIG. 1, once the cart 11 is properly positioned, the robotic arms 12 may insert the steerable endoscope 13 into the patient robotically, manually, or a combination thereof. As shown, the steerable endoscope 13 may comprise at least two telescoping parts, such as an inner leader (e.g., a scope) portion and an outer sheath portion, each portion coupled to a separate instrument driver from the set of instrument drivers 28, each instrument driver coupled to the distal end of an individual robotic arm. This linear arrangement of the instrument drivers 28, which facilitates coaxially aligning the leader portion with the sheath portion, creates a “virtual rail” 29 that may be repositioned in space by manipulating the one or more robotic arms 12 into different angles and/or positions. The virtual rails described herein are depicted in the Figures using dashed lines, and accordingly the dashed lines do not depict any physical structure of the system. Translation of the instrument drivers 28 along the virtual rail 29 telescopes the inner leader portion relative to the outer sheath portion or advances or retracts the endoscope 13 from the patient. The angle of the virtual rail 29 may be adjusted, translated, and pivoted based on clinical application or physician preference. For example, in bronchoscopy, the angle and position of the virtual rail 29 as shown represents a compromise between providing physician access to the endoscope 13 while minimizing friction that results from bending the endoscope 13 into the patient's mouth.

The endoscope 13 may be directed down the patient's trachea and lungs after insertion using precise commands from the robotic system until reaching the target destination or operative site. In order to enhance navigation through the patient's lung network and/or reach the desired target, the endoscope 13 may be manipulated to telescopically extend the inner leader portion from the outer sheath portion to obtain enhanced articulation and greater bend radius. The use of separate instrument drivers 28 also allows the leader portion and sheath portion to be driven independent of each other.

For example, the endoscope 13 may be directed to deliver a biopsy needle to a target, such as, for example, a lesion or nodule within the lungs of a patient. The needle may be deployed down a working channel that runs the length of the endoscope to obtain a tissue sample to be analyzed by a pathologist. Depending on the pathology results, additional tools may be deployed down the working channel of the endoscope for additional biopsies. After identifying a nodule to be malignant, the endoscope 13 may endoscopically deliver tools to resect the potentially cancerous tissue. In some instances, diagnostic and therapeutic treatments can be delivered in separate procedures. In those circumstances, the endoscope 13 may also be used to deliver a fiducial to “mark” the location of the target nodule as well. In other instances, diagnostic and therapeutic treatments may be delivered during the same procedure.

The system 10 may also include a movable tower 30, which may be connected via support cables to the cart 11 to provide support for controls, electronics, fluidics, optics, sensors, and/or power to the cart 11. Placing such functionality in the tower 30 allows for a smaller form factor cart 11 that may be more easily adjusted and/or repositioned by an operating physician and his/her staff. Additionally, the division of functionality between the cart/table and the support tower 30 reduces operating room clutter and facilitates improving clinical workflow. While the cart 11 may be positioned close to the patient, the tower 30 may be stowed in a remote location to stay out of the way during a procedure.

In support of the robotic systems described above, the tower 30 may include component(s) of a computer-based control system that stores computer program instructions, for example, within a non-transitory computer-readable storage medium such as a persistent magnetic storage drive, solid state drive, etc. The execution of those instructions, whether the execution occurs in the tower 30 or the cart 11, may control the entire system or sub-system(s) thereof. For example, when executed by a processor of the computer system, the instructions may cause the components of the robotics system to actuate the relevant carriages and arm mounts, actuate the robotics arms, and control the medical instruments. For example, in response to receiving the control signal, the motors in the joints of the robotics arms may position the arms into a certain posture.

The tower 30 may also include a pump, flow meter, valve control, and/or fluid access in order to provide controlled irrigation and aspiration capabilities to the system that may be deployed through the endoscope 13. These components may also be controlled using the computer system of tower 30. In some embodiments, irrigation and aspiration capabilities may be delivered directly to the endoscope 13 through separate cable(s).

The tower 30 may include a voltage and surge protector designed to provide filtered and protected electrical power to the cart 11, thereby avoiding placement of a power transformer and other auxiliary power components in the cart 11, resulting in a smaller, more moveable cart 11.

The tower 30 may also include support equipment for the sensors deployed throughout the robotic system 10. For example, the tower 30 may include opto-electronics equipment for detecting, receiving, and processing data received from the optical sensors or cameras throughout the robotic system 10. In combination with the control system, such opto-electronics equipment may be used to generate real-time images for display in any number of consoles deployed throughout the system, including in the tower 30. Similarly, the tower 30 may also include an electronic subsystem for receiving and processing signals received from deployed electromagnetic (EM) sensors. The tower 30 may also be used to house and position an EM field generator for detection by EM sensors in or on the medical instrument.

The tower 30 may also include a console 31 in addition to other consoles available in the rest of the system, e.g., console mounted on top of the cart. The console 31 may include a user interface and a display screen, such as a touchscreen, for the physician operator. Consoles in system 10 are generally designed to provide both robotic controls as well as pre-operative and real-time information of the procedure, such as navigational and localization information of the endoscope 13. When the console 31 is not the only console available to the physician, it may be used by a second operator, such as a nurse, to monitor the health or vitals of the patient and the operation of system, as well as provide procedure-specific data, such as navigational and localization information. In other embodiments, the console 30 is housed in a body that is separate from the tower 30.

The tower 30 may be coupled to the cart 11 and endoscope 13 through one or more cables or connections (not shown). In some embodiments, the support functionality from the tower 30 may be provided through a single cable to the cart 11, simplifying and de-cluttering the operating room. In other embodiments, specific functionality may be coupled in separate cabling and connections. For example, while power may be provided through a single power cable to the cart, the support for controls, optics, fluidics, and/or navigation may be provided through a separate cable.

FIG. 2 provides a detailed illustration of an embodiment of the cart from the cart-based robotically-enabled system shown in FIG. 1. The cart 11 generally includes an elongated support structure 14 (often referred to as a “column”), a cart base 15, and a console 16 at the top of the column 14. The column 14 may include one or more carriages, such as a carriage 17 (alternatively “arm support”) for supporting the deployment of one or more robotic arms 12 (three shown in FIG. 2). The carriage 17 may include individually configurable arm mounts that rotate along a perpendicular axis to adjust the base of the robotic arms 12 for better positioning relative to the patient. The carriage 17 also includes a carriage interface 19 that allows the carriage 17 to vertically translate along the column 14.

The carriage interface 19 is connected to the column 14 through slots, such as slot 20, that are positioned on opposite sides of the column 14 to guide the vertical translation of the carriage 17. The slot 20 contains a vertical translation interface to position and hold the carriage at various vertical heights relative to the cart base 15. Vertical translation of the carriage 17 allows the cart 11 to adjust the reach of the robotic arms 12 to meet a variety of table heights, patient sizes, and physician preferences. Similarly, the individually configurable arm mounts on the carriage 17 allow the robotic arm base 21 of robotic arms 12 to be angled in a variety of configurations.

In some embodiments, the slot 20 may be supplemented with slot covers that are flush and parallel to the slot surface to prevent dirt and fluid ingress into the internal chambers of the column 14 and the vertical translation interface as the carriage 17 vertically translates. The slot covers may be deployed through pairs of spring spools positioned near the vertical top and bottom of the slot 20. The covers are coiled within the spools until deployed to extend and retract from their coiled state as the carriage 17 vertically translates up and down. The spring-loading of the spools provides force to retract the cover into a spool when carriage 17 translates towards the spool, while also maintaining a tight seal when the carriage 17 translates away from the spool. The covers may be connected to the carriage 17 using, for example, brackets in the carriage interface 19 to ensure proper extension and retraction of the cover as the carriage 17 translates.

The column 14 may internally comprise mechanisms, such as gears and motors, that are designed to use a vertically aligned lead screw to translate the carriage 17 in a mechanized fashion in response to control signals generated in response to user inputs, e.g., inputs from the console 16.

The robotic arms 12 may generally comprise robotic arm bases 21 and end effectors 22, separated by a series of linkages 23 that are connected by a series of joints 24, each joint comprising an independent actuator, each actuator comprising an independently controllable motor. Each independently controllable joint represents an independent degree of freedom available to the robotic arm. Each of the arms 12 have seven joints, and thus provide seven degrees of freedom. A multitude of joints result in a multitude of degrees of freedom, allowing for “redundant” degrees of freedom. Redundant degrees of freedom allow the robotic arms 12 to position their respective end effectors 22 at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions.

The cart base 15 balances the weight of the column 14, carriage 17, and arms 12 over the floor. Accordingly, the cart base 15 houses heavier components, such as electronics, motors, power supply, as well as components that either enable movement and/or immobilize the cart. For example, the cart base 15 includes rollable wheel-shaped casters 25 that allow for the cart to easily move around the room prior to a procedure. After reaching the appropriate position, the casters 25 may be immobilized using wheel locks to hold the cart 11 in place during the procedure.

Positioned at the vertical end of column 14, the console 16 allows for both a user interface for receiving user input and a display screen (or a dual-purpose device such as, for example, a touchscreen 26) to provide the physician user with both pre-operative and intra-operative data. Potential pre-operative data on the touchscreen 26 may include pre-operative plans, navigation and mapping data derived from pre-operative computerized tomography (CT) scans, and/or notes from pre-operative patient interviews. Intra-operative data on display may include optical information provided from the tool, sensor and coordinate information from sensors, as well as vital patient statistics, such as respiration, heart rate, and/or pulse. The console 16 may be positioned and tilted to allow a physician to access the console from the side of the column 14 opposite carriage 17. From this position, the physician may view the console 16, robotic arms 12, and patient while operating the console 16 from behind the cart 11. As shown, the console 16 also includes a handle 27 to assist with maneuvering and stabilizing cart 11.

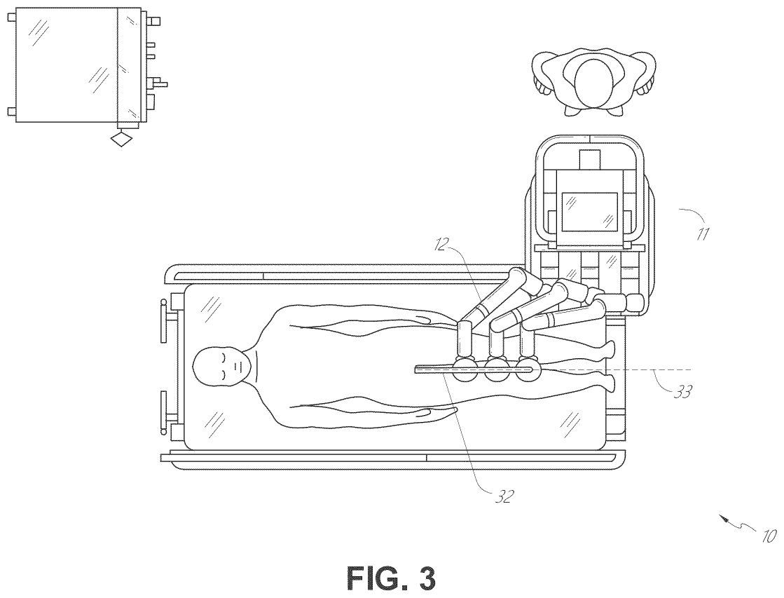

FIG. 3 illustrates an embodiment of a robotically-enabled system 10 arranged for ureteroscopy. In a ureteroscopic procedure, the cart 11 may be positioned to deliver a ureteroscope 32, a procedure-specific endoscope designed to traverse a patient's urethra and ureter, to the lower abdominal area of the patient. In a ureteroscopy, it may be desirable for the ureteroscope 32 to be directly aligned with the patient's urethra to reduce friction and forces on the sensitive anatomy in the area. As shown, the cart 11 may be aligned at the foot of the table to allow the robotic arms 12 to position the ureteroscope 32 for direct linear access to the patient's urethra. From the foot of the table, the robotic arms 12 may insert the ureteroscope 32 along the virtual rail 33 directly into the patient's lower abdomen through the urethra.

After insertion into the urethra, using similar control techniques as in bronchoscopy, the ureteroscope 32 may be navigated into the bladder, ureters, and/or kidneys for diagnostic and/or therapeutic applications. For example, the ureteroscope 32 may be directed into the ureter and kidneys to break up kidney stone build up using a laser or ultrasonic lithotripsy device deployed down the working channel of the ureteroscope 32. After lithotripsy is complete, the resulting stone fragments may be removed using baskets deployed down the ureteroscope 32.

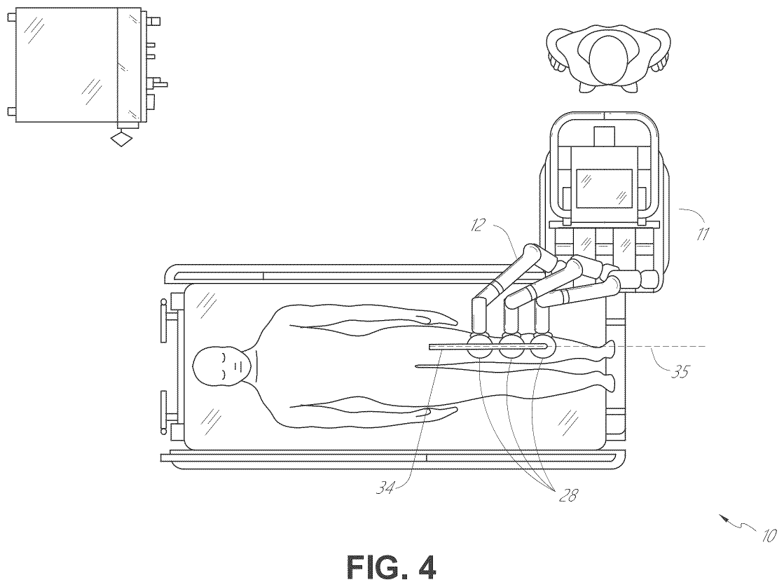

FIG. 4 illustrates an embodiment of a robotically-enabled system similarly arranged for a vascular procedure. In a vascular procedure, the system 10 may be configured such that the cart 11 may deliver a medical instrument 34, such as a steerable catheter, to an access point in the femoral artery in the patient's leg. The femoral artery presents both a larger diameter for navigation as well as a relatively less circuitous and tortuous path to the patient's heart, which simplifies navigation. As in a ureteroscopic procedure, the cart 11 may be positioned towards the patient's legs and lower abdomen to allow the robotic arms 12 to provide a virtual rail 35 with direct linear access to the femoral artery access point in the patient's thigh/hip region. After insertion into the artery, the medical instrument 34 may be directed and inserted by translating the instrument drivers 28. Alternatively, the cart may be positioned around the patient's upper abdomen in order to reach alternative vascular access points, such as, for example, the carotid and brachial arteries near the shoulder and wrist.

### B. Robotic System—Table.

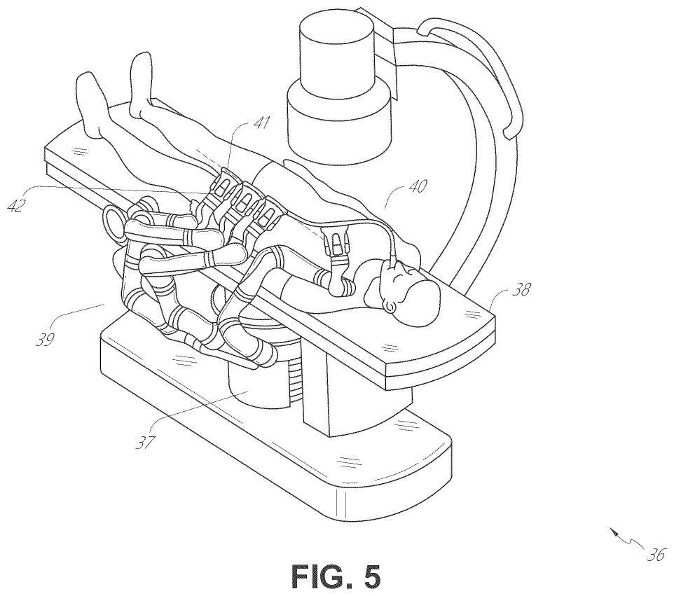

Embodiments of the robotically-enabled medical system may also incorporate the patient's table. Incorporation of the table reduces the amount of capital equipment within the operating room by removing the cart, which allows greater access to the patient. FIG. 5 illustrates an embodiment of such a robotically-enabled system arranged for a bronchoscopy procedure. System 36 includes a support structure or column 37 for supporting platform 38 (shown as a “table” or “bed”) over the floor. Much like in the cart-based systems, the end effectors of the robotic arms 39 of the system 36 comprise instrument drivers 42 that are designed to manipulate an elongated medical instrument, such as a bronchoscope 40 in FIG. 5, through or along a virtual rail 41 formed from the linear alignment of the instrument drivers 42. In practice, a C-arm for providing fluoroscopic imaging may be positioned over the patient's upper abdominal area by placing the emitter and detector around table 38.

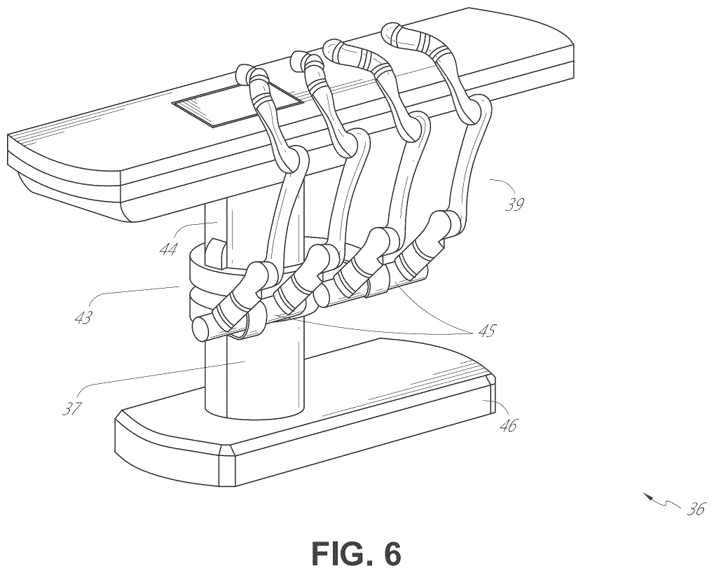

FIG. 6 provides an alternative view of the system 36 without the patient and medical instrument for discussion purposes. As shown, the column 37 may include one or more carriages 43 shown as ring-shaped in the system 36, from which the one or more robotic arms 39 may be based. The carriages 43 may translate along a vertical column interface 44 that runs the length of the column 37 to provide different vantage points from which the robotic arms 39 may be positioned to reach the patient. The carriage(s) 43 may rotate around the column 37 using a mechanical motor positioned within the column 37 to allow the robotic arms 39 to have access to multiples sides of the table 38, such as, for example, both sides of the patient. In embodiments with multiple carriages, the carriages may be individually positioned on the column and may translate and/or rotate independent of the other carriages. While carriages 43 need not surround the column 37 or even be circular, the ring-shape as shown facilitates rotation of the carriages 43 around the column 37 while maintaining structural balance. Rotation and translation of the carriages 43 allows the system to align the medical instruments, such as endoscopes and laparoscopes, into different access points on the patient. In other embodiments (not shown), the system 36 can include a patient table or bed with adjustable arm supports in the form of bars or rails extending alongside it. One or more robotic arms 39 (e.g., via a shoulder with an elbow joint) can be attached to the adjustable arm supports, which can be vertically adjusted. By providing vertical adjustment, the robotic arms 39 are advantageously capable of being stowed compactly beneath the patient table or bed, and subsequently raised during a procedure.

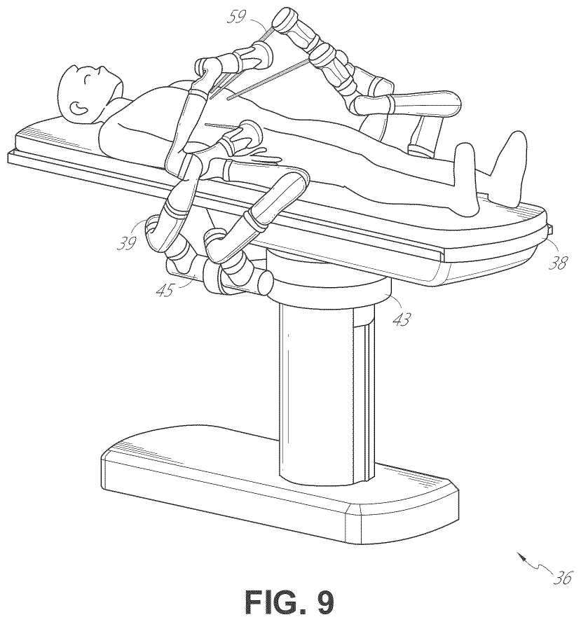

The arms 39 may be mounted on the carriages through a set of arm mounts 45 comprising a series of joints that may individually rotate and/or telescopically extend to provide additional configurability to the robotic arms 39. Additionally, the arm mounts 45 may be positioned on the carriages 43 such that, when the carriages 43 are appropriately rotated, the arm mounts 45 may be positioned on either the same side of table 38 (as shown in FIG. 6), on opposite sides of table 38 (as shown in FIG. 9), or on adjacent sides of the table 38 (not shown).

The column 37 structurally provides support for the table 38, and a path for vertical translation of the carriages. Internally, the column 37 may be equipped with lead screws for guiding vertical translation of the carriages, and motors to mechanize the translation of said carriages based the lead screws. The column 37 may also convey power and control signals to the carriage 43 and robotic arms 39 mounted thereon.

The table base 46 serves a similar function as the cart base 15 in cart 11 shown in FIG. 2, housing heavier components to balance the table/bed 38, the column 37, the carriages 43, and the robotic arms 39. The table base 46 may also incorporate rigid casters to provide stability during procedures. Deployed from the bottom of the table base 46, the casters may extend in opposite directions on both sides of the base 46 and retract when the system 36 needs to be moved.

Continuing with FIG. 6, the system 36 may also include a tower (not shown) that divides the functionality of system 36 between table and tower to reduce the form factor and bulk of the table. As in earlier disclosed embodiments, the tower may provide a variety of support functionalities to table, such as processing, computing, and control capabilities, power, fluidics, and/or optical and sensor processing. The tower may also be movable to be positioned away from the patient to improve physician access and de-clutter the operating room. Additionally, placing components in the tower allows for more storage space in the table base for potential stowage of the robotic arms. The tower may also include a master controller or console that provides both a user interface for user input, such as keyboard and/or pendant, as well as a display screen (or touchscreen) for pre-operative and intra-operative information, such as real-time imaging, navigation, and tracking information. In some embodiments, the tower may also contain holders for gas tanks to be used for insufflation.

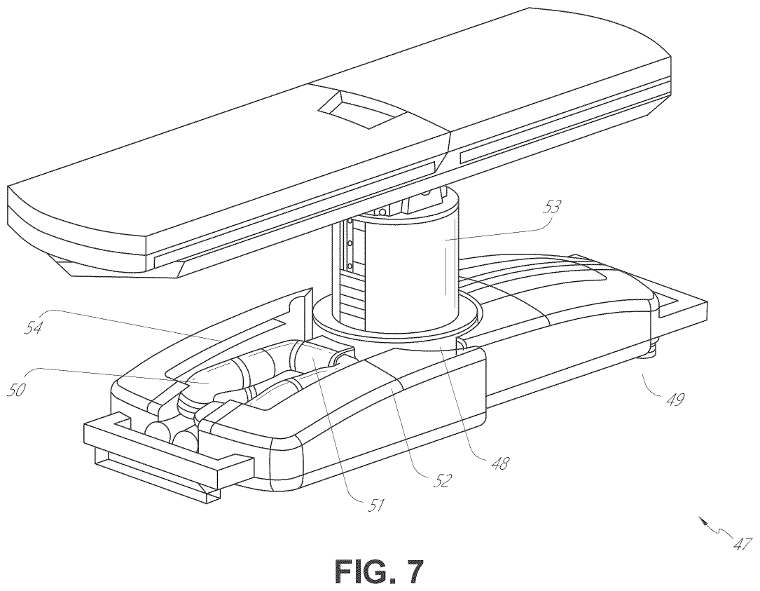

In some embodiments, a table base may stow and store the robotic arms when not in use. FIG. 7 illustrates a system 47 that stows robotic arms in an embodiment of the table-based system. In system 47, carriages 48 may be vertically translated into base 49 to stow robotic arms 50, arm mounts 51, and the carriages 48 within the base 49. Base covers 52 may be translated and retracted open to deploy the carriages 48, arm mounts 51, and arms 50 around column 53, and closed to stow to protect them when not in use. The base covers 52 may be sealed with a membrane 54 along the edges of its opening to prevent dirt and fluid ingress when closed.

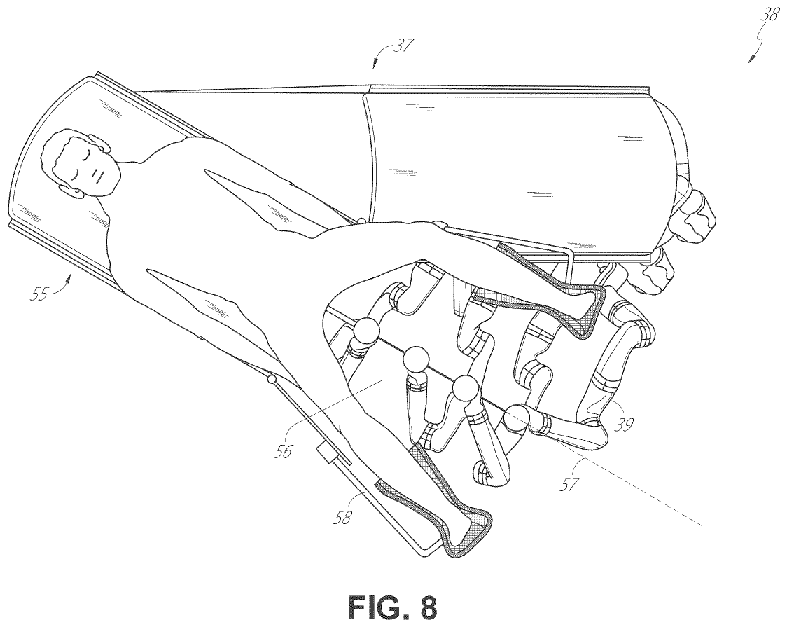

FIG. 8 illustrates an embodiment of a robotically-enabled table-based system configured for a ureteroscopy procedure. In a ureteroscopy, the table 38 may include a swivel portion 55 for positioning a patient off-angle from the column 37 and table base 46. The swivel portion 55 may rotate or pivot around a pivot point (e.g., located below the patient's head) in order to position the bottom portion of the swivel portion 55 away from the column 37. For example, the pivoting of the swivel portion 55 allows a C-arm (not shown) to be positioned over the patient's lower abdomen without competing for space with the column (not shown) below table 38. By rotating the carriage 35 (not shown) around the column 37, the robotic arms 39 may directly insert a ureteroscope 56 along a virtual rail 57 into the patient's groin area to reach the urethra. In a ureteroscopy, stirrups 58 may also be fixed to the swivel portion 55 of the table 38 to support the position of the patient's legs during the procedure and allow clear access to the patient's groin area.

In a laparoscopic procedure, through small incision(s) in the patient's abdominal wall, minimally invasive instruments may be inserted into the patient's anatomy. In some embodiments, the minimally invasive instruments comprise an elongated rigid member, such as a shaft, which is used to access anatomy within the patient. After inflation of the patient's abdominal cavity, the instruments may be directed to perform surgical or medical tasks, such as grasping, cutting, ablating, suturing, etc. In some embodiments, the instruments can comprise a scope, such as a laparoscope. FIG. 9 illustrates an embodiment of a robotically-enabled table-based system configured for a laparoscopic procedure. As shown in FIG. 9, the carriages 43 of the system 36 may be rotated and vertically adjusted to position pairs of the robotic arms 39 on opposite sides of the table 38, such that instruments 59 may be positioned using the arm mounts 45 to be passed through minimal incisions on both sides of the patient to reach his/her abdominal cavity.

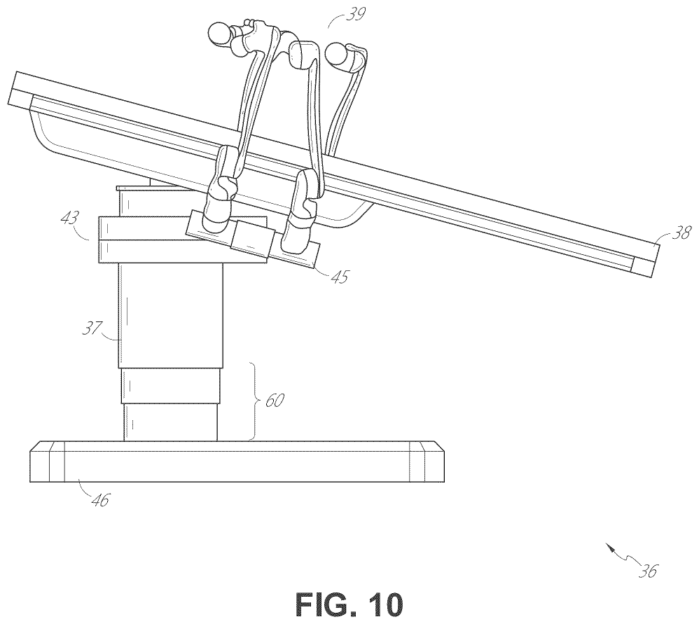

To accommodate laparoscopic procedures, the robotically-enabled table system may also tilt the platform to a desired angle. FIG. 10 illustrates an embodiment of the robotically-enabled medical system with pitch or tilt adjustment. As shown in FIG. 10, the system 36 may accommodate tilt of the table 38 to position one portion of the table at a greater distance from the floor than the other. Additionally, the arm mounts 45 may rotate to match the tilt such that the arms 39 maintain the same planar relationship with table 38. To accommodate steeper angles, the column 37 may also include telescoping portions 60 that allow vertical extension of column 37 to keep the table 38 from touching the floor or colliding with base 46.

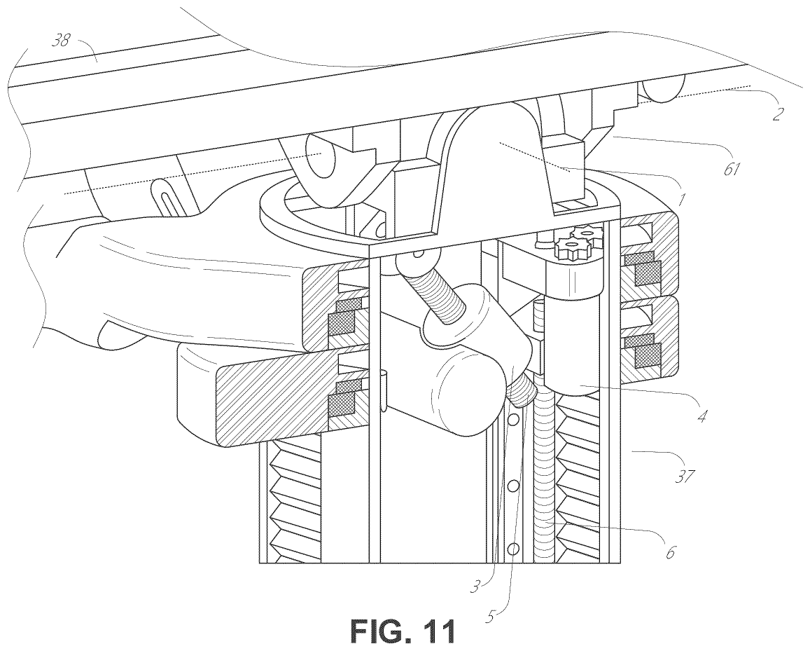

FIG. 11 provides a detailed illustration of the interface between the table 38 and the column 37. Pitch rotation mechanism 61 may be configured to alter the pitch angle of the table 38 relative to the column 37 in multiple degrees of freedom. The pitch rotation mechanism 61 may be enabled by the positioning of orthogonal axes 1, 2 at the column-table interface, each axis actuated by a separate motor 3, 4 responsive to an electrical pitch angle command. Rotation along one screw 5 would enable tilt adjustments in one axis 1, while rotation along the other screw 6 would enable tilt adjustments along the other axis 2. In some embodiments, a ball joint can be used to alter the pitch angle of the table 38 relative to the column 37 in multiple degrees of freedom.

For example, pitch adjustments are particularly useful when trying to position the table in a Trendelenburg position, i.e., position the patient's lower abdomen at a higher position from the floor than the patient's lower abdomen, for lower abdominal surgery. The Trendelenburg position causes the patient's internal organs to slide towards his/her upper abdomen through the force of gravity, clearing out the abdominal cavity for minimally invasive tools to enter and perform lower abdominal surgical or medical procedures, such as laparoscopic prostatectomy.

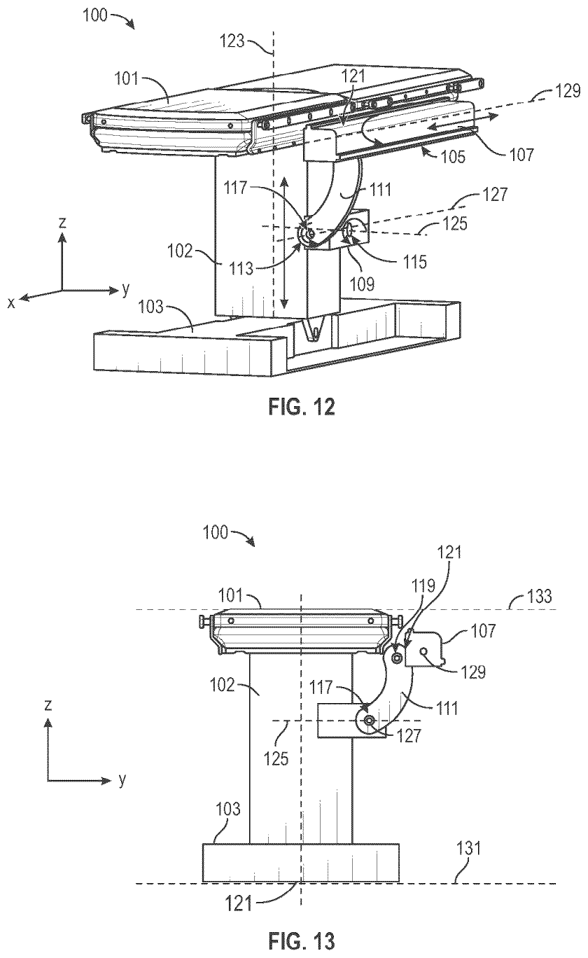

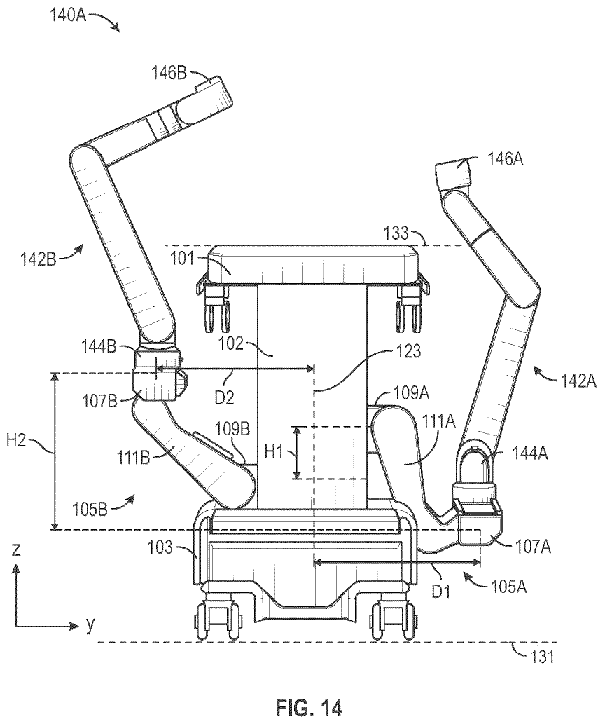

FIGS. 12 and 13 illustrate isometric and end views of another embodiment of a table-based surgical robotics system 100. The surgical robotics system 100 includes one or more adjustable arm supports 105 that can be configured to support one or more robotic arms (see, for example, FIG. 14) relative to a table 101. In the illustrated embodiment, a single adjustable arm support 105 is shown, though an additional arm support can be provided on an opposite side of the table 101. The adjustable arm support 105 can be configured so that it can move relative to the table 101 to adjust and/or vary the position of the adjustable arm support 105 and/or any robotic arms mounted thereto relative to the table 101. For example, the adjustable arm support 105 may be adjusted one or more degrees of freedom relative to the table 101. The adjustable arm support 105 provides high versatility to the system 100, including the ability to easily stow the one or more adjustable arm supports 105 and any robotics arms attached thereto beneath the table 101. The adjustable arm support 105 can be elevated from the stowed position to a position below an upper surface of the table 101. In other embodiments, the adjustable arm support 105 can be elevated from the stowed position to a position above an upper surface of the table 101.

The adjustable arm support 105 can provide several degrees of freedom, including lift, lateral translation, tilt, etc. In the illustrated embodiment of FIGS. 12 and 13, the arm support 105 is configured with four degrees of freedom, which are illustrated with arrows in FIG. 12. A first degree of freedom allows for adjustment of the adjustable arm support 105 in the z-direction (“Z-lift”). For example, the adjustable arm support 105 can include a carriage 109 configured to move up or down along or relative to a column 102 supporting the table 101. A second degree of freedom can allow the adjustable arm support 105 to tilt. For example, the adjustable arm support 105 can include a rotary joint, which can allow the adjustable arm support 105 to be aligned with the bed in a Trendelenburg position. A third degree of freedom can allow the adjustable arm support 105 to “pivot up,” which can be used to adjust a distance between a side of the table 101 and the adjustable arm support 105. A fourth degree of freedom can permit translation of the adjustable arm support 105 along a longitudinal length of the table.

The surgical robotics system 100 in FIGS. 12 and 13 can comprise a table supported by a column 102 that is mounted to a base 103. The base 103 and the column 102 support the table 101 relative to a support surface. A floor axis 131 and a support axis 133 are shown in FIG. 13.

The adjustable arm support 105 can be mounted to the column 102. In other embodiments, the arm support 105 can be mounted to the table 101 or base 103. The adjustable arm support 105 can include a carriage 109, a bar or rail connector 111 and a bar or rail 107. In some embodiments, one or more robotic arms mounted to the rail 107 can translate and move relative to one another.

The carriage 109 can be attached to the column 102 by a first joint 113, which allows the carriage 109 to move relative to the column 102 (e.g., such as up and down a first or vertical axis 123). The first joint 113 can provide the first degree of freedom (“Z-lift”) to the adjustable arm support 105. The adjustable arm support 105 can include a second joint 115, which provides the second degree of freedom (tilt) for the adjustable arm support 105. The adjustable arm support 105 can include a third joint 117, which can provide the third degree of freedom (“pivot up”) for the adjustable arm support 105. An additional joint 119 (shown in FIG. 13) can be provided that mechanically constrains the third joint 117 to maintain an orientation of the rail 107 as the rail connector 111 is rotated about a third axis 127. The adjustable arm support 105 can include a fourth joint 121, which can provide a fourth degree of freedom (translation) for the adjustable arm support 105 along a fourth axis 129.

FIG. 14 illustrates an end view of the surgical robotics system 140A with two adjustable arm supports 105A, 105B mounted on opposite sides of a table 101. A first robotic arm 142A is attached to the bar or rail 107A of the first adjustable arm support 105B. The first robotic arm 142A includes a base 144A attached to the rail 107A. The distal end of the first robotic arm 142A includes an instrument drive mechanism 146A that can attach to one or more robotic medical instruments or tools. Similarly, the second robotic arm 142B includes a base 144B attached to the rail 107B. The distal end of the second robotic arm 142B includes an instrument drive mechanism 146B. The instrument drive mechanism 146B can be configured to attach to one or more robotic medical instruments or tools.

In some embodiments, one or more of the robotic arms 142A, 142B comprises an arm with seven or more degrees of freedom. In some embodiments, one or more of the robotic arms 142A, 142B can include eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and base 144A, 144B (1-degree of freedom including translation). In some embodiments, the insertion degree of freedom can be provided by the robotic arm 142A, 142B, while in other embodiments, the instrument itself provides insertion via an instrument-based insertion architecture.

### C. Instrument Driver & Interface.

The end effectors of the system's robotic arms comprise (i) an instrument driver (alternatively referred to as “instrument drive mechanism” or “instrument device manipulator”) that incorporate electro-mechanical means for actuating the medical instrument and (ii) a removable or detachable medical instrument, which may be devoid of any electro-mechanical components, such as motors. This dichotomy may be driven by the need to sterilize medical instruments used in medical procedures, and the inability to adequately sterilize expensive capital equipment due to their intricate mechanical assemblies and sensitive electronics. Accordingly, the medical instruments may be designed to be detached, removed, and interchanged from the instrument driver (and thus the system) for individual sterilization or disposal by the physician or the physician's staff. In contrast, the instrument drivers need not be changed or sterilized, and may be draped for protection.

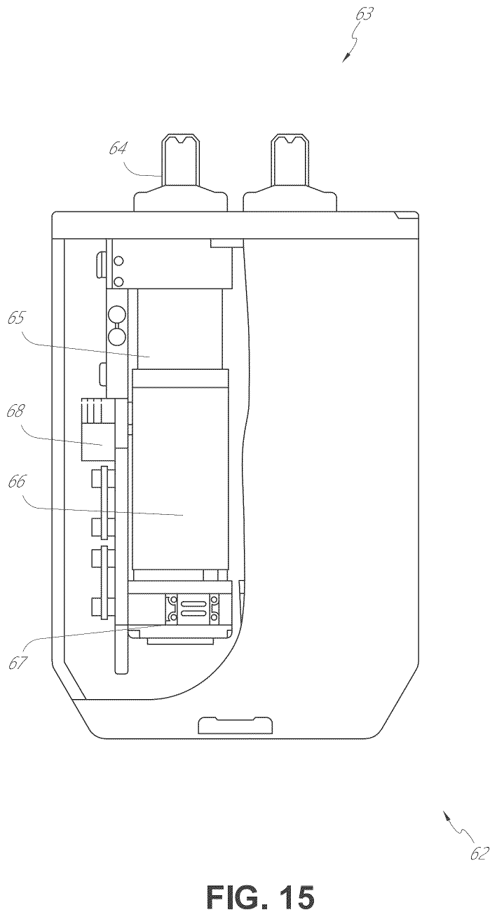

FIG. 15 illustrates an example instrument driver. Positioned at the distal end of a robotic arm, instrument driver 62 comprises of one or more drive units 63 arranged with parallel axes to provide controlled torque to a medical instrument via drive shafts 64. Each drive unit 63 comprises an individual drive shaft 64 for interacting with the instrument, a gear head 65 for converting the motor shaft rotation to a desired torque, a motor 66 for generating the drive torque, an encoder 67 to measure the speed of the motor shaft and provide feedback to the control circuitry, and control circuitry 68 for receiving control signals and actuating the drive unit. Each drive unit 63 being independent controlled and motorized, the instrument driver 62 may provide multiple (four as shown in FIG. 15) independent drive outputs to the medical instrument. In operation, the control circuitry 68 would receive a control signal, transmit a motor signal to the motor 66, compare the resulting motor speed as measured by the encoder 67 with the desired speed, and modulate the motor signal to generate the desired torque.

For procedures that require a sterile environment, the robotic system may incorporate a drive interface, such as a sterile adapter connected to a sterile drape, that sits between the instrument driver and the medical instrument. The chief purpose of the sterile adapter is to transfer angular motion from the drive shafts of the instrument driver to the drive inputs of the instrument while maintaining physical separation, and thus sterility, between the drive shafts and drive inputs. Accordingly, an example sterile adapter may comprise of a series of rotational inputs and outputs intended to be mated with the drive shafts of the instrument driver and drive inputs on the instrument. Connected to the sterile adapter, the sterile drape, comprised of a thin, flexible material such as transparent or translucent plastic, is designed to cover the capital equipment, such as the instrument driver, robotic arm, and cart (in a cart-based system) or table (in a table-based system). Use of the drape would allow the capital equipment to be positioned proximate to the patient while still being located in an area not requiring sterilization (i.e., non-sterile field). On the other side of the sterile drape, the medical instrument may interface with the patient in an area requiring sterilization (i.e., sterile field).

### D. Medical Instrument.

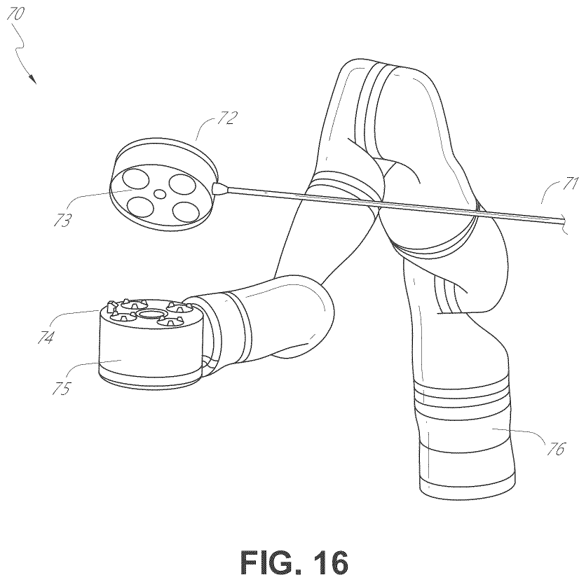

FIG. 16 illustrates an example medical instrument with a paired instrument driver. Like other instruments designed for use with a robotic system, medical instrument 70 comprises an elongated shaft 71 (or elongate body) and an instrument base 72. The instrument base 72, also referred to as an “instrument handle” due to its intended design for manual interaction by the physician, may generally comprise rotatable drive inputs 73, e.g., receptacles, pulleys or spools, that are designed to be mated with drive outputs 74 that extend through a drive interface on instrument driver 75 at the distal end of robotic arm 76. When physically connected, latched, and/or coupled, the mated drive inputs 73 of instrument base 72 may share axes of rotation with the drive outputs 74 in the instrument driver 75 to allow the transfer of torque from drive outputs 74 to drive inputs 73. In some embodiments, the drive outputs 74 may comprise splines that are designed to mate with receptacles on the drive inputs 73.

The elongated shaft 71 is designed to be delivered through either an anatomical opening or lumen, e.g., as in endoscopy, or a minimally invasive incision, e.g., as in laparoscopy. The elongated shaft 71 may be either flexible (e.g., having properties similar to an endoscope) or rigid (e.g., having properties similar to a laparoscope) or contain a customized combination of both flexible and rigid portions. When designed for laparoscopy, the distal end of a rigid elongated shaft may be connected to an end effector extending from a jointed wrist formed from a clevis with at least one degree of freedom and a surgical tool or medical instrument, such as, for example, a grasper or scissors, that may be actuated based on force from the tendons as the drive inputs rotate in response to torque received from the drive outputs 74 of the instrument driver 75. When designed for endoscopy, the distal end of a flexible elongated shaft may include a steerable or controllable bending section that may be articulated and bent based on torque received from the drive outputs 74 of the instrument driver 75.

Torque from the instrument driver 75 is transmitted down the elongated shaft 71 using tendons along the shaft 71. These individual tendons, such as pull wires, may be individually anchored to individual drive inputs 73 within the instrument handle 72. From the handle 72, the tendons are directed down one or more pull lumens along the elongated shaft 71 and anchored at the distal portion of the elongated shaft 71, or in the wrist at the distal portion of the elongated shaft. During a surgical procedure, such as a laparoscopic, endoscopic or hybrid procedure, these tendons may be coupled to a distally mounted end effector, such as a wrist, grasper, or scissor. Under such an arrangement, torque exerted on drive inputs 73 would transfer tension to the tendon, thereby causing the end effector to actuate in some way. In some embodiments, during a surgical procedure, the tendon may cause a joint to rotate about an axis, thereby causing the end effector to move in one direction or another. Alternatively, the tendon may be connected to one or more jaws of a grasper at distal end of the elongated shaft 71, where tension from the tendon cause the grasper to close.

In endoscopy, the tendons may be coupled to a bending or articulating section positioned along the elongated shaft 71 (e.g., at the distal end) via adhesive, control ring, or other mechanical fixation. When fixedly attached to the distal end of a bending section, torque exerted on drive inputs 73 would be transmitted down the tendons, causing the softer, bending section (sometimes referred to as the articulable section or region) to bend or articulate. Along the non-bending sections, it may be advantageous to spiral or helix the individual pull lumens that direct the individual tendons along (or inside) the walls of the endoscope shaft to balance the radial forces that result from tension in the pull wires. The angle of the spiraling and/or spacing there between may be altered or engineered for specific purposes, wherein tighter spiraling exhibits lesser shaft compression under load forces, while lower amounts of spiraling results in greater shaft compression under load forces, but also exhibits limits bending. On the other end of the spectrum, the pull lumens may be directed parallel to the longitudinal axis of the elongated shaft 71 to allow for controlled articulation in the desired bending or articulable sections.

In endoscopy, the elongated shaft 71 houses a number of components to assist with the robotic procedure. The shaft may comprise of a working channel for deploying surgical tools (or medical instruments), irrigation, and/or aspiration to the operative region at the distal end of the shaft 71. The shaft 71 may also accommodate wires and/or optical fibers to transfer signals to/from an optical assembly at the distal tip, which may include of an optical camera. The shaft 71 may also accommodate optical fibers to carry light from proximally-located light sources, such as light emitting diodes, to the distal end of the shaft.

At the distal end of the instrument 70, the distal tip may also comprise the opening of a working channel for delivering tools for diagnostic and/or therapy, irrigation, and aspiration to an operative site. The distal tip may also include a port for a camera, such as a fiberscope or a digital camera, to capture images of an internal anatomical space. Relatedly, the distal tip may also include ports for light sources for illuminating the anatomical space when using the camera.

In the example of FIG. 16, the drive shaft axes, and thus the drive input axes, are orthogonal to the axis of the elongated shaft. This arrangement, however, complicates roll capabilities for the elongated shaft 71. Rolling the elongated shaft 71 along its axis while keeping the drive inputs 73 static results in undesirable tangling of the tendons as they extend off the drive inputs 73 and enter pull lumens within the elongated shaft 71. The resulting entanglement of such tendons may disrupt any control algorithms intended to predict movement of the flexible elongated shaft during an endoscopic procedure.

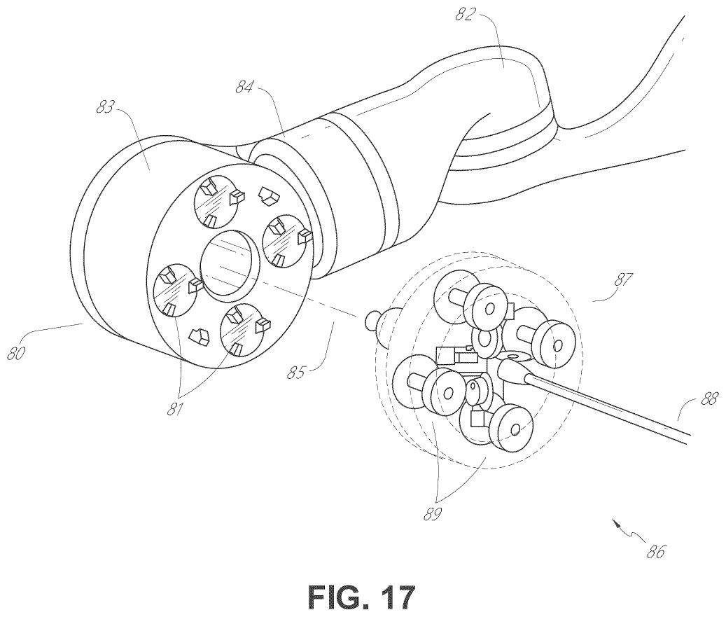

FIG. 17 illustrates an alternative design for an instrument driver and instrument where the axes of the drive units are parallel to the axis of the elongated shaft of the instrument. As shown, a circular instrument driver 80 comprises four drive units with their drive outputs 81 aligned in parallel at the end of a robotic arm 82. The drive units, and their respective drive outputs 81, are housed in a rotational assembly 83 of the instrument driver 80 that is driven by one of the drive units within the assembly 83. In response to torque provided by the rotational drive unit, the rotational assembly 83 rotates along a circular bearing that connects the rotational assembly 83 to the non-rotational portion 84 of the instrument driver. Power and controls signals may be communicated from the non-rotational portion 84 of the instrument driver 80 to the rotational assembly 83 through electrical contacts may be maintained through rotation by a brushed slip ring connection (not shown). In other embodiments, the rotational assembly 83 may be responsive to a separate drive unit that is integrated into the non-rotatable portion 84, and thus not in parallel to the other drive units. The rotational mechanism 83 allows the instrument driver 80 to rotate the drive units, and their respective drive outputs 81, as a single unit around an instrument driver axis 85.

Like earlier disclosed embodiments, an instrument 86 may comprise an elongated shaft portion 88 and an instrument base 87 (shown with a transparent external skin for discussion purposes) comprising a plurality of drive inputs 89 (such as receptacles, pulleys, and spools) that are configured to receive the drive outputs 81 in the instrument driver 80. Unlike prior disclosed embodiments, instrument shaft 88 extends from the center of instrument base 87 with an axis substantially parallel to the axes of the drive inputs 89, rather than orthogonal as in the design of FIG. 16.

When coupled to the rotational assembly 83 of the instrument driver 80, the medical instrument 86, comprising instrument base 87 and instrument shaft 88, rotates in combination with the rotational assembly 83 about the instrument driver axis 85. Since the instrument shaft 88 is positioned at the center of instrument base 87, the instrument shaft 88 is coaxial with instrument driver axis 85 when attached. Thus, rotation of the rotational assembly 83 causes the instrument shaft 88 to rotate about its own longitudinal axis. Moreover, as the instrument base 87 rotates with the instrument shaft 88, any tendons connected to the drive inputs 89 in the instrument base 87 are not tangled during rotation. Accordingly, the parallelism of the axes of the drive outputs 81, drive inputs 89, and instrument shaft 88 allows for the shaft rotation without tangling any control tendons.

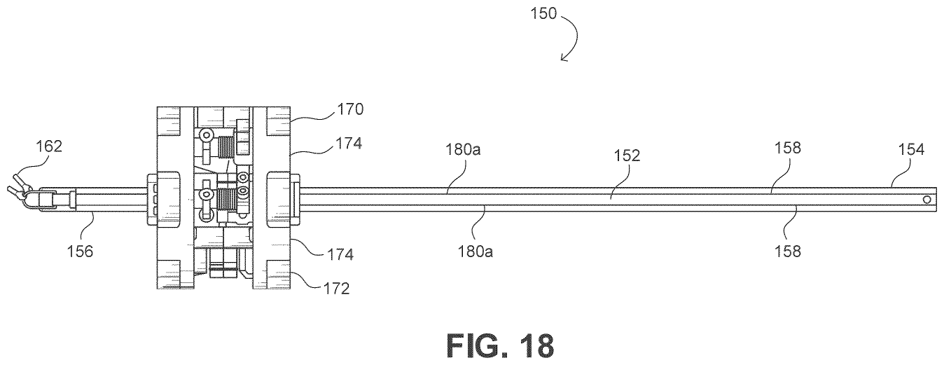

FIG. 18 illustrates an instrument having an instrument based insertion architecture in accordance with some embodiments. The instrument 150 can be coupled to any of the instrument drivers discussed above. The instrument 150 comprises an elongated shaft 152, an end effector 162 connected to the shaft 152, and a handle 170 coupled to the shaft 152. The elongated shaft 152 comprises a tubular member having a proximal portion 154 and a distal portion 156. The elongated shaft 152 comprises one or more channels or grooves 158 along its outer surface. The grooves 158 are configured to receive one or more wires or cables 180 therethrough. One or more cables 180 thus run along an outer surface of the elongated shaft 152. In other embodiments, cables 180 can also run through the elongated shaft 152. Manipulation of the one or more cables 180 (e.g., via an instrument driver) results in actuation of the end effector 162.

The instrument handle 170, which may also be referred to as an instrument base, may generally comprise an attachment interface 172 having one or more mechanical inputs 174, e.g., receptacles, pulleys or spools, that are designed to be reciprocally mated with one or more torque couplers on an attachment surface of an instrument driver.

In some embodiments, the instrument 150 comprises a series of pulleys or cables that enable the elongated shaft 152 to translate relative to the handle 170. In other words, the instrument 150 itself comprises an instrument-based insertion architecture that accommodates insertion of the instrument, thereby minimizing the reliance on a robot arm to provide insertion of the instrument 150. In other embodiments, a robotic arm can be largely responsible for instrument insertion.

### E. Controller.

Any of the robotic systems described herein can include an input device or controller for manipulating an instrument attached to a robotic arm. In some embodiments, the controller can be coupled (e.g., communicatively, electronically, electrically, wirelessly and/or mechanically) with an instrument such that manipulation of the controller causes a corresponding manipulation of the instrument e.g., via master slave control.

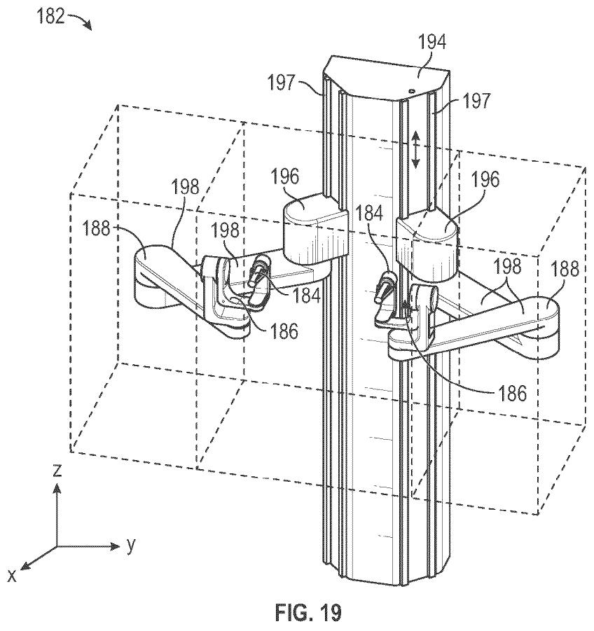

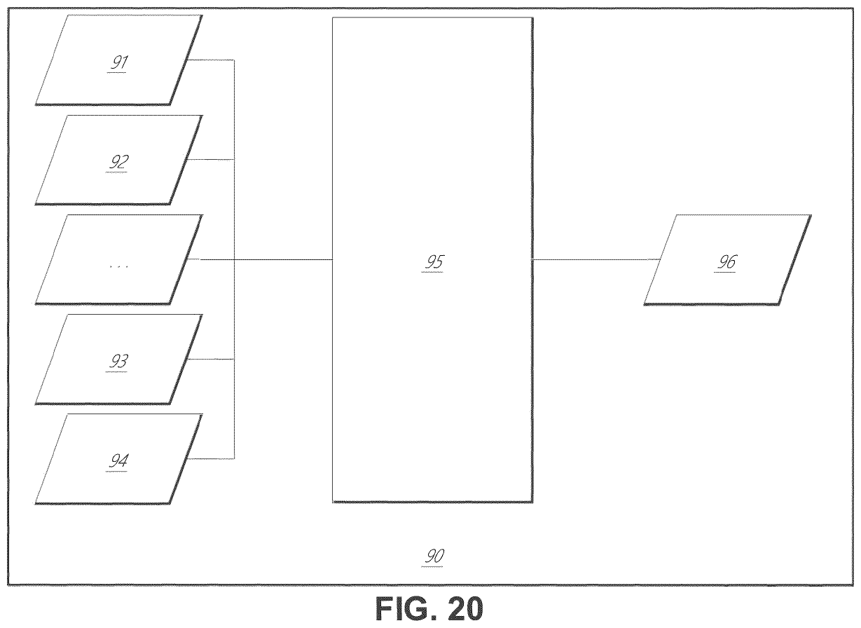

FIG. 19 is a perspective view of an embodiment of a controller 182. In the present embodiment, the controller 182 comprises a hybrid controller that can have both impedance and admittance control. In other embodiments, the controller 182 can utilize just impedance or passive control. In other embodiments, the controller 182 can utilize just admittance control. By being a hybrid controller, the controller 182 advantageously can have a lower perceived inertia while in use.

In the illustrated embodiment, the controller 182 is configured to allow manipulation of two medical instruments, and includes two handles 184. Each of the handles 184 is connected to a gimbal 186. Each gimbal 186 is connected to a positioning platform 188.

As shown in FIG. 19, each positioning platform 188 includes a SCARA arm (selective compliance assembly robot arm) 198 coupled to a column 194 by a prismatic joint 196. The prismatic joints 196 are configured to translate along the column 194 (e.g., along rails 197) to allow each of the handles 184 to be translated in the z-direction, providing a first degree of freedom. The SCARA arm 198 is configured to allow motion of the handle 184 in an x-y plane, providing two additional degrees of freedom.

In some embodiments, one or more load cells are positioned in the controller. For example, in some embodiments, a load cell (not shown) is positioned in the body of each of the gimbals 186. By providing a load cell, portions of the controller 182 are capable of operating under admittance control, thereby advantageously reducing the perceived inertia of the controller while in use. In some embodiments, the positioning platform 188 is configured for admittance control, while the gimbal 186 is configured for impedance control. In other embodiments, the gimbal 186 is configured for admittance control, while the positioning platform 188 is configured for impedance control. Accordingly, for some embodiments, the translational or positional degrees of freedom of the positioning platform 188 can rely on admittance control, while the rotational degrees of freedom of the gimbal 186 rely on impedance control.

### F. Navigation and Control.

Traditional endoscopy may involve the use of fluoroscopy (e.g., as may be delivered through a C-arm) and other forms of radiation-based imaging modalities to provide endoluminal guidance to an operator physician. In contrast, the robotic systems contemplated by this disclosure can provide for non-radiation-based navigational and localization means to reduce physician exposure to radiation and reduce the amount of equipment within the operating room. As used herein, the term “localization” may refer to determining and/or monitoring the position of objects in a reference coordinate system. Technologies such as pre-operative mapping, computer vision, real-time EM tracking, and robot command data may be used individually or in combination to achieve a radiation-free operating environment. In other cases, where radiation-based imaging modalities are still used, the pre-operative mapping, computer vision, real-time EM tracking, and robot command data may be used individually or in combination to improve upon the information obtained solely through radiation-based imaging modalities.

FIG. 20 is a block diagram illustrating a localization system 90 that estimates a location of one or more elements of the robotic system, such as the location of the instrument, in accordance to an example embodiment. The localization system 90 may be a set of one or more computer devices configured to execute one or more instructions. The computer devices may be embodied by a processor (or processors) and computer-readable memory in one or more components discussed above. By way of example and not limitation, the computer devices may be in the tower 30 shown in FIG. 1, the cart shown in FIGS. 1-4, the beds shown in FIGS. 5-14, etc.

As shown in FIG. 20, the localization system 90 may include a localization module 95 that processes input data 91-94 to generate location data 96 for the distal tip of a medical instrument. The location data 96 may be data or logic that represents a location and/or orientation of the distal end of the instrument relative to a frame of reference. The frame of reference can be a frame of reference relative to the anatomy of the patient or to a known object, such as an EM field generator (see discussion below for the EM field generator).

The various input data 91-94 are now described in greater detail. Pre-operative mapping may be accomplished through the use of the collection of low dose CT scans. Pre-operative CT scans are reconstructed into three-dimensional images, which are visualized, e.g. as “slices” of a cutaway view of the patient's internal anatomy. When analyzed in the aggregate, image-based models for anatomical cavities, spaces and structures of the patient's anatomy, such as a patient lung network, may be generated. Techniques such as center-line geometry may be determined and approximated from the CT images to develop a three-dimensional volume of the patient's anatomy, referred to as model data 91 (also referred to as “preoperative model data” when generated using only preoperative CT scans). The use of center-line geometry is discussed in U.S. patent application Ser. No. 14/523,760, the contents of which are herein incorporated in its entirety. Network topological models may also be derived from the CT-images, and are particularly appropriate for bronchoscopy.

In some embodiments, the instrument may be equipped with a camera to provide vision data 92. The localization module 95 may process the vision data to enable one or more vision-based location tracking. For example, the preoperative model data may be used in conjunction with the vision data 92 to enable computer vision-based tracking of the medical instrument (e.g., an endoscope or an instrument advance through a working channel of the endoscope). For example, using the preoperative model data 91, the robotic system may generate a library of expected endoscopic images from the model based on the expected path of travel of the endoscope, each image linked to a location within the model. Intra-operatively, this library may be referenced by the robotic system in order to compare real-time images captured at the camera (e.g., a camera at a distal end of the endoscope) to those in the image library to assist localization.

Other computer vision-based tracking techniques use feature tracking to determine motion of the camera, and thus the endoscope. Some features of the localization module 95 may identify circular geometries in the preoperative model data 91 that correspond to anatomical lumens and track the change of those geometries to determine which anatomical lumen was selected, as well as the relative rotational and/or translational motion of the camera. Use of a topological map may further enhance vision-based algorithms or techniques.

Optical flow, another computer vision-based technique, may analyze the displacement and translation of image pixels in a video sequence in the vision data 92 to infer camera movement. Examples of optical flow techniques may include motion detection, object segmentation calculations, luminance, motion compensated encoding, stereo disparity measurement, etc. Through the comparison of multiple frames over multiple iterations, movement and location of the camera (and thus the endoscope) may be determined.

The localization module 95 may use real-time EM tracking to generate a real-time location of the endoscope in a global coordinate system that may be registered to the patient's anatomy, represented by the preoperative model. In EM tracking, an EM sensor (or tracker) comprising of one or more sensor coils embedded in one or more locations and orientations in a medical instrument (e.g., an endoscopic tool) measures the variation in the EM field created by one or more static EM field generators positioned at a known location. The location information detected by the EM sensors is stored as EM data 93. The EM field generator (or transmitter), may be placed close to the patient to create a low intensity magnetic field that the embedded sensor may detect. The magnetic field induces small currents in the sensor coils of the EM sensor, which may be analyzed to determine the distance and angle between the EM sensor and the EM field generator. These distances and orientations may be intra-operatively “registered” to the patient anatomy (e.g., the preoperative model) in order to determine the geometric transformation that aligns a single location in the coordinate system with a position in the pre-operative model of the patient's anatomy. Once registered, an embedded EM tracker in one or more positions of the medical instrument (e.g., the distal tip of an endoscope) may provide real-time indications of the progression of the medical instrument through the patient's anatomy.

Robotic command and kinematics data 94 may also be used by the localization module 95 to provide localization data 96 for the robotic system. Device pitch and yaw resulting from articulation commands may be determined during pre-operative calibration. Intra-operatively, these calibration measurements may be used in combination with known insertion depth information to estimate the position of the instrument. Alternatively, these calculations may be analyzed in combination with EM, vision, and/or topological modeling to estimate the position of the medical instrument within the network.

As FIG. 17 shows, a number of other input data can be used by the localization module 95. For example, although not shown in FIG. 17, an instrument utilizing shape-sensing fiber can provide shape data that the localization module 95 can use to determine the location and shape of the instrument.

The localization module 95 may use the input data 91-94 in combination(s). In some cases, such a combination may use a probabilistic approach where the localization module 95 assigns a confidence weight to the location determined from each of the input data 91-94. Thus, where the EM data may not be reliable (as may be the case where there is EM interference) the confidence of the location determined by the EM data 93 can be decrease and the localization module 95 may rely more heavily on the vision data 92 and/or the robotic command and kinematics data 94.

As discussed above, the robotic systems discussed herein may be designed to incorporate a combination of one or more of the technologies above. The robotic system's computer-based control system, based in the tower, bed and/or cart, may store computer program instructions, for example, within a non-transitory computer-readable storage medium such as a persistent magnetic storage drive, solid state drive, or the like, that, upon execution, cause the system to receive and analyze sensor data and user commands, generate control signals throughout the system, and display the navigational and localization data, such as the position of the instrument within the global coordinate system, anatomical map, etc.

### 2. User Interfaces for Navigation in Medical Procedures

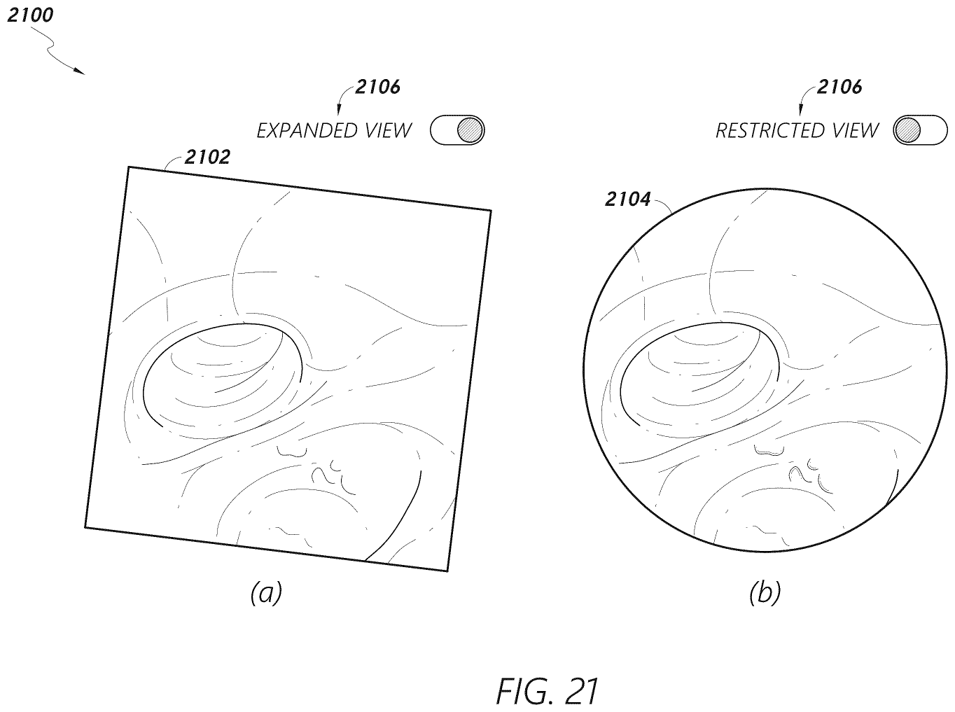

FIG. 21 illustrates an example of a graphical user interface (GUI) 2100 which provides either or both of an expanded field-of-view 2102 and a restricted field-of-view 2104 of an intraoperative image captured by an endoscope. On the left half of FIG. 21, the GUI 2100 presents the expanded field-of-view 2102 and on the right half of FIG. 21, the GUI 2100 presents the restricted field-of-view 2104 on a viewer/display. Both views 2102, 2104 may present the intraoperative image captured at an anatomical site.

In some embodiments, the GUI 2100 may present the image in both the expanded field-of-view 2102 and the restricted field-of-view 2104 side by side as shown. In other embodiments, a GUI may include only one selected view of the two views 2102, 2104. For instance, the GUI 2100 may present only the restricted field-of-view 2104, provided as a circular cropped shape.

The expanded field-of-view 2102 can present a superset of visuals presented in the restricted field-of-view 2104 to provide a better recognition of position and orientation of the endoscope in association with surrounding features presented in the expanded field-of-view 2102 but not in the restricted field-of-view 2104. In some embodiments, the expanded field-of-view 2102 can be an uncropped endoscopic feed captured by an imaging device of the endoscope with its full field-of-view (FOV). Shape of the image is not limited to the square shape of the expanded field-of-view 2102 as shown but any shape provided by the imaging device.

The expanded field-of-view 2102 of the live endoscopic view can provide operators with more camera data on the screen as they are navigating during the procedure. The conventional live scope view is a circular cutout of the entire endoscope camera; however, the camera typically collects more data than is currently displayed to the operator. Providing the operator with more of the available image captured by an imaging device may be beneficial in scenarios where an airway or other anatomy is visible but is previously being cropped out of view. In some embodiments, the operator has the ability to toggle the cutout off in order to get the full image from the imaging device. This allow improved navigation and the ability to better correct a trajectory of the endoscope.

In some embodiments, the expanded field-of-view 2102 may supplement its captured endoscopic feed with a predicted or otherwise simulated portions to expand beyond its full field-of-view and present uncaptured features in the expanded field-of-view 2102. For instance, based on a current position and orientation of the endoscope in relation to a target anatomical site, various image generation algorithms can process preoperative model data (e.g., CT scan data) to predict or simulate features surrounding, but not visible in, the endoscopic feed. As another instance, artificial intelligence or a trained machine learning algorithm can be used to predict or simulate such features. Image portions predicted or simulated may be provided with lighter brightness, less contrast, or less emphasis so as to (i) distinguish between actual captured portions and generated image portions and (ii) to not distract an operator.

The restricted field-of-view 2104 can present a subset of visuals presented in the expanded field-of-view 2102 to (i) better focus on features near the center of the endoscopic feed or (ii) to provide a more instinctive navigational guidance to an operator of the endoscope. For example, a circular view as shown in the restricted field-of-view 2104 may be more readily associated with, often, a circular shape of the endoscope such that the operator can more instinctively navigate the endoscope based on visuals of the restricted field-of-view 2104. It is noted that the expanded field-of-view 2102 and the restricted field-of-view 2104 are not limited by their respective shapes but by being a superset or a subset of the respective field-of-views they present. That is, although the expanded field-of-view 2102 is illustrated as a square field-of-view and the restricted field-of-view 2104 is shown as a circular field-of-view, it should be appreciated that any other shapes can be used. For example, any other shape such as any polygon, curved shapes, or any other shape may be applied as a mask or a filter to the expanded field-of-view 2102 and the restricted field-of-view 2104 as long as the restricted field-of-view 2104 is a subset of the expanded field-of-view 2102.

In some implementations where the two field-of-views 2102, 2104 are not presented together but only one field-of-view is presented, an operator may manually toggle between the expanded field-of-view 2102 and the restricted field-of-view 2104. The toggling may be performed by manually selecting a toggle button 2106. Although the toggle button 2106 is shown as a slidable switch, the toggle button 2106 may be any selectable graphical control element that can capture a selection or non-selection of a field-of-view such as a radio button, a checkbox, or the like. In some embodiments, the toggle button 2106 may not be a graphical control element but a physical control element of the medical system. Some example of control elements include physical buttons, touch sensitive buttons, touch screen icons, joysticks, foot pedals, and other elements of input devices that may be used to provide inputs to the system.

In some embodiments, toggling between the expanded field-of-view 2102 and the restricted field-of-view 2104—and vice versa—may be done automatically based on a detected condition. For example, a detected condition may include detecting an obstruction or blockage in the navigation path of the endoscope, detecting mucus in the navigation path of the endoscope, or detecting an adjacent airway that is blocking navigation or that may be of interest to the operator and/or is otherwise not being presently displayed in the restricted field-of-view 2104. In addition, toggling between the expanded and restricted field-of-views 2102, 2104 may depend on the location of a target or lesion, a position and orientation of a surgical needle with respect to the location of the target or lesion—for example, as the needle is closer to approaching the lesion, an expanded field-of-view 2102 may automatically be toggled on. Further, airway configurations, airway sizes, and airway positions may influence the automatic toggling on and off of the restricted and expanded field-of-views 2102, 2104.

It should be appreciated that machine learning and artificial intelligence may be utilized to train an algorithm to predict or detect other circumstances intraoperatively where an expanded field-of-view 2102 would be useful to a physician and toggle the expanded field-of-view 2102 in response. For example, it can be predicted or determined that certain positions, orientations, or junctions within a luminal network may pose some challenge (e.g., a particularly confusing set of branches) for correct navigation and, when it is determined that the endoscope is at the position, orientation, or junction, the expanded field-of-view 2102 may be toggled on automatically. In some embodiments, a recommendation (e.g., a tip text or an icon) to turn on the expanded field-of-view 2102 may be presented instead of automatically turning on the expanded field-of-view 210. Similarly, in some implementations, the expanded field-of-view 2102 may be toggled off automatically when the endoscope has moved away from the position, orientation, or junction.

In some embodiments, one or more visual indicators or effects may be applied to the expanded field-of-view 2102 to distinguish its expanded image portions from restricted image portions of the restricted field-of-view 2104. For example, an outline of a shape of the restricted field-of-view 2104 can be overlaid on the expanded field-of-view 2102 when the expanded field-of-view 2102 is toggled on. Referring to the GUI 2100, for example, a circular outline of the restricted field-of-view 2104 may be overlaid on the expanded field-of-view. As another example, the expanded image portions can be shaded, greyed out, blurred, provided with lighter colors, or the like.

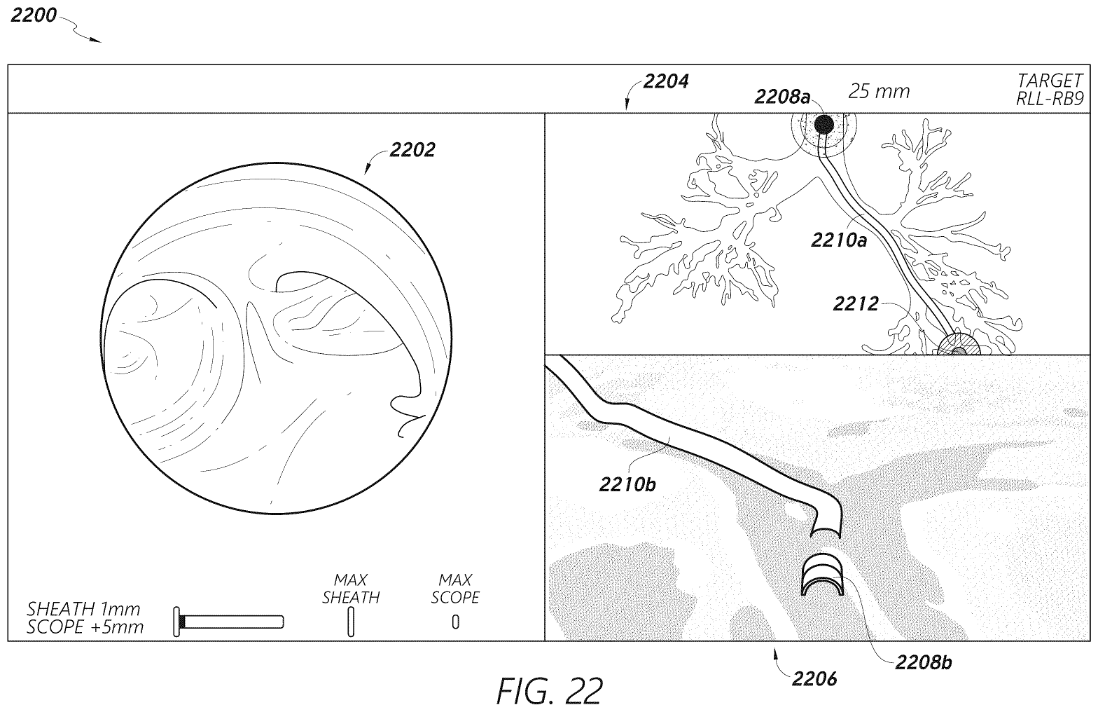

FIG. 22 illustrates an example of a GUI 2200 including various views. A live endoscopic view 2202, presented on the left half of the GUI 2200, may default to the restricted field-of-view 2104 of FIG. 21. At the top right is a path view 2204 and at the bottom right is a CT view 2206. As shown, the path view 2204 can present a view of a 3D model of a luminal network (e.g., a lung) and the CT view 2206 can present a view of a CT image. The path view 2204 may use, as its 3D model, a model generated based on a plurality of CT images. For instance, the 3D model may be preoperative model data generated based on model data 91 of FIG. 20. As shown, the path view 2204 and the CT view 2206 may present a tip indicator 2208a-b, a path indicator 2210a-b, and a target indicator 2212 to assist navigation of the endoscope.

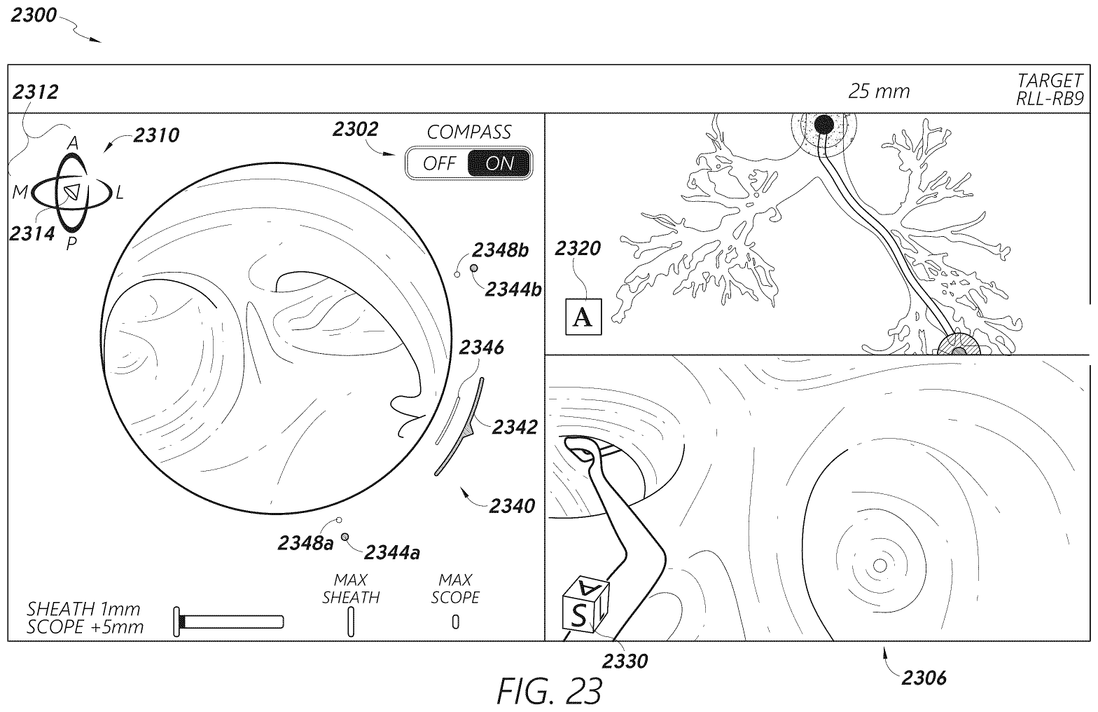

FIG. 23 illustrates an example of a GUI 2300 including a plurality of orientation indicators. The GUI 2300 can include a selectable user interface (UI) element (e.g., a toggle button) 2302 that turns on or off the presentation of the orientation indicators. When the UI element 2302 is set to present the orientation indicators, each of the presented orientation indicators can be displayed to inform an operator of a current orientation of the endoscope. In this and other examples described herein, the orientation of the scope can be determined based on one or more tracking systems associated with the scope that can detect orientation of scope as it traverses the patient anatomy. Examples of tracking systems include electromagnetic (EM) tracking systems, fiber optic shape sensors, image sensors, and other systems that include sensors to determine the orientation of the scope (or tip of the scope) relative to the patient anatomy. Tracking systems may also use a combination of sensor inputs to determine the orientation of the sensor within the patient anatomy. One example of a tracking system is described above with reference to FIG. 20, and can be used to determine the orientation of the scope to facilitate the orientation indicators or compasses described herein.

A first orientation indicator 2310 may be presented outside or adjacent to an endoscopic view (e.g., the endoscopic view 2202 of FIG. 22). The endoscopic view can be a live image feed of the patient anatomy captured by the scope as it traverses the patient anatomy. The first orientation indicator 2310 may provide reference axis/plane 2312 regarding one or more anatomical directions, such as Anterior/Posterior axis (denoted with “A” and “P”) or Medial/Lateral axis (denoted with “M” and “L”). The first orientation indicator 2310 may additionally provide a direction indicator 2314, which may be 2D such as an arrow or a directional needle or a 3D such as a cone as shown, to inform an orientation of the endoscope associated with the endoscopic view. For instance, in the GUI 2300, the first orientation indicator 2310 can inform an operator that top of the endoscopic view is toward Anterior, bottom is toward Posterior, left is toward Medial, and right is toward Lateral. In some other embodiments, when the endoscope adjusts its orientation, the direction indicator 2314, the reference axis/plane 2312, or both may adjust by a corresponding amount in the display. For example, when the endoscope “rolls” along its longitudinal axis, the direction indicator 2314 may roll by the corresponding roll amount in the display while the reference axis/plane 2312 remain fixed.

The reference axis/plane 2312, and one or more anatomical directions thereof, may be determined during preoperation or intraoperation. For example, preoperatively, a known placement of an endoscope in relation to a known positioning of a patient on a supporting platform (e.g., the supporting platform 38 of FIG. 5) may inform the GUI 2300 of a default set of reference axis/plane 2312. As another example, preoperatively or intraoperatively, an operator may manually input or adjust reference axis/plane 2312. As yet another example, preoperatively or intraoperatively, the reference axis/plane 2312 may be automatically determined by performing a calibration procedure with the endoscope.

An example calibration or registration procedure can include advancement of the endoscope into then out of at least two known reference branches of a luminal network. This procedure can facilitate registration of a tracking system's reference frame to the reference frame of the patient anatomy. For example, the operator may advance a bronchoscope into the left bronchus, retract out of the left bronchus, and advance the bronchoscope into the right bronchus. This calibration procedure can provide multiple reference points which may be mapped to at least one axis, provide a zero point on the axis, and anatomical directions based on the zero point. Use of the calibration procedure may advantageously enable exact determination of the reference axis/plane 2312, and anatomical directions thereof, without errors often associated with default or manual reference configurations.

A second orientation indicator 2320 illustrates a 2D orientation indicator, represented as a box. The second orientation indicator 2320 can inform an operator that a path view (e.g., the path view 2204 of FIG. 22) is seen from Anterior toward Posterior by presenting “A” while hiding “P.” In some implementations, the second orientation indicator 2320 may be tilted by a corresponding amount when the path view as shown is rotated clockwise or counter-clockwise. Although a box is shown, it is noted that any indicative shapes may be used.

A third orientation indicator 2330 illustrates a 3D orientation indicator, represented as a cube. The third orientation indicator 2330 can inform an operator that a simulated view 2306, predicted based on a preoperative model, is seen from Anterior toward Posterior and from Superior to Inferior by presenting an upside-down “A” and an upright “S.” Further, a slight showing of “L” for Lateral informs the operator that the simulated view 2306 is seen from an angle that is slightly Lateral toward Medial orientation. Accordingly, the angling of each faces of the cube and which letters are visible in what orientations can reflect an orientation of an endoscope in the simulated view 2306. Although a cube is shown, it is noted that any indicative objects may be used.

As the orientation indicators 2310, 2320, 2330 and their respective views illustrate, each view may be presented with a corresponding orientation indicator that independently informs orientation of an image presented in the view. For instance, the second orientation indicator 2320 reflects a first orientation of the path view while the third orientation indicator 2330 reflects a second orientation for the simulated view 2330 that is different from the first orientation.

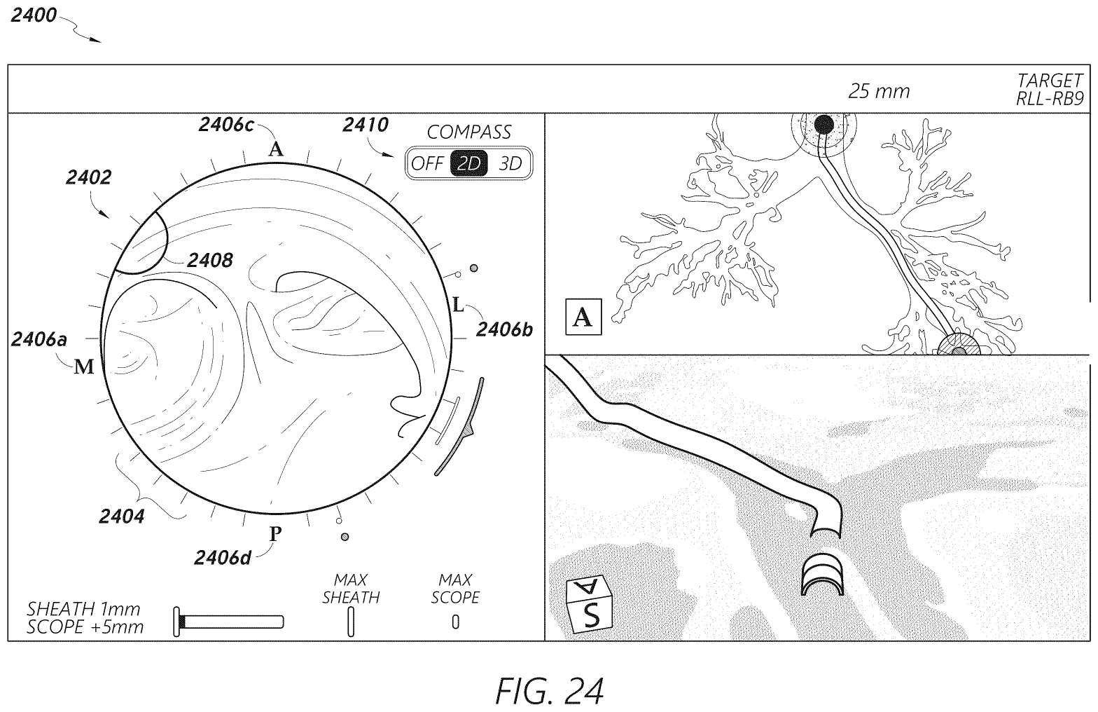

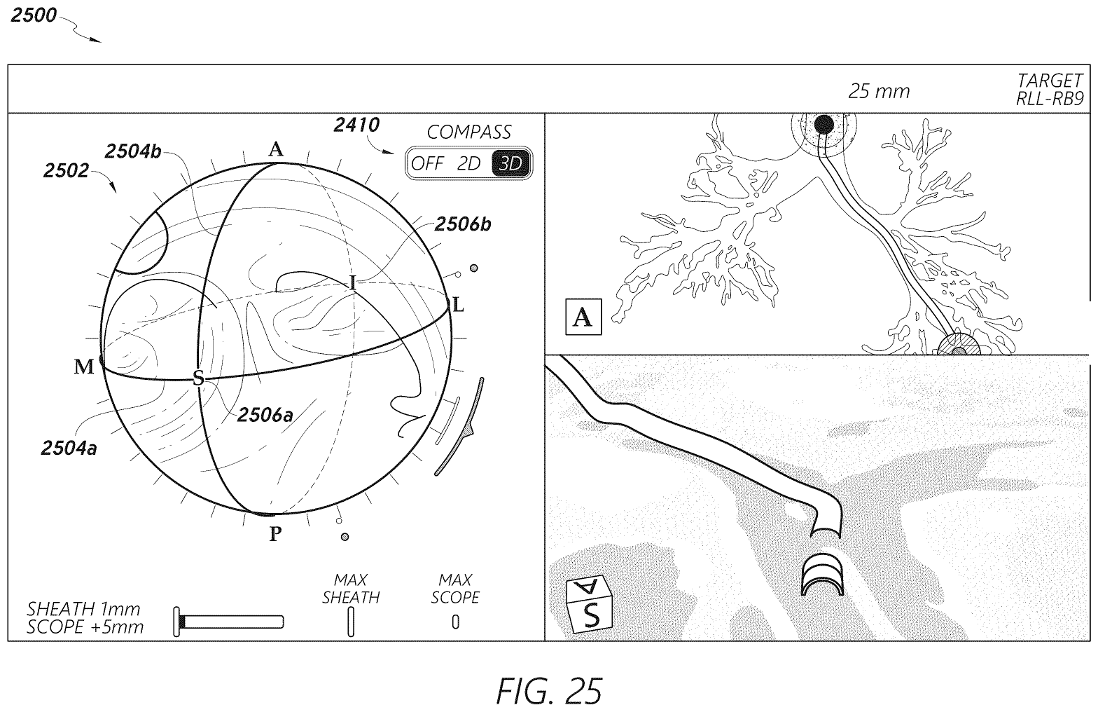

In the present disclosure, an orientation indicator, including the described orientation indicators 2310, 2320, 2330, may be referred as a “compass.” Although structure and elements of the orientation indicator may differ from a traditional physical compass, the term is to be interpreted broadly here to include UI elements that can help an operator determine orientation of an endoscope with respect to a shown view. In some embodiments, the whole or a portion of a compass may be presented adjacent to or overlaid inside a peripheral boundary of the endoscopic view so as to provide instinctive association between the compass and the endoscopic view as will be shown and described in greater detail with respect to FIGS. 24-25.

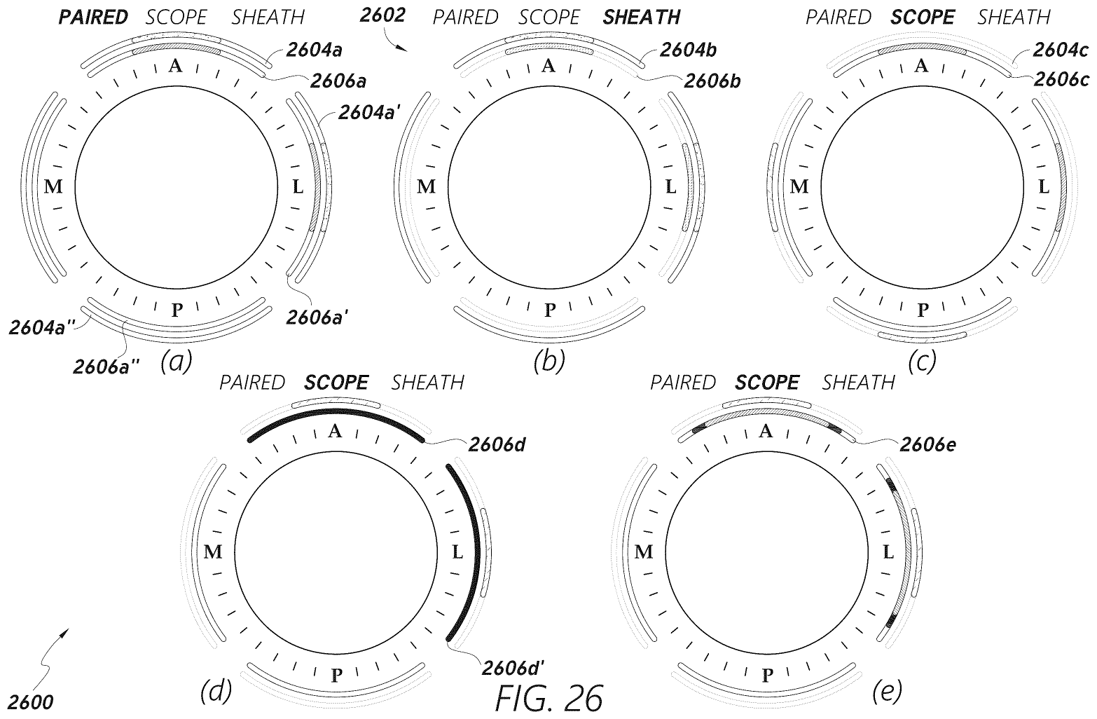

In some embodiments, an articulation indicator 2340 may be presented adjacent to or within the endoscopic view. The articulation indicator 2340 can indicate which anatomical direction the endoscope is currently articulated to or articulating toward. Where the endoscope has multiple separately articulable components, such as a sheath portion and a leader portion, a separate articulation indicator 2342, 2346 may be provided for each articulable component. As shown, the separate articulation indicator 2342, 2346 may be presented as a variable length arc that has a center positioned toward an articulation direction and a length corresponding to a magnitude of articulation. Maximum available articulation can be indicated with a first set of end points 2344a-b for the sheath articulation indicator 2342 and a second set of end points 2348a-b for the leader articulation indicator 2346. Although variable length arcs are shown as examples, other graphical elements, such as a variable thickness arrows, may represent articulation vectors (i.e., direction and magnitude of articulation). Another embodiment of the articulation indicator 2340 is described in greater detail with respect to FIG. 26.

FIG. 24 illustrates an example of a GUI 2400 including an example of a 2D compass 2402. The 2D compass 2402 may surround periphery of an endoscopic view (e.g., the endoscopic view 2202 of FIG. 22) to provide an operator with a better sense of direction as they are navigating intraoperatively. The 2D compass 2402 may include letters at different positions along the boundary of the compass to indicate corresponding anatomical directions such as Superior/Inferior, Anterior/Posterior, Medial/Lateral, Proximal/Distal, Central/Peripheral, Superficial/Deep, Dorsal/Ventral, or the like. In the example 2D compass 2402, anatomical directions of Anterior/Posterior and Medial/Lateral are provided with, respectively, letters A/P and M/L, referred herein as anatomical direction indicators 2406a-d. Each pair of the anatomical direction indicators 2406a-d may be presented at opposing ends of the peripheral boundary. The 2D compass 2402 may provide angular index/scale 2404 around the peripheral boundary of the endoscopic view to indicate regular angular intervals of 5 degrees, 10 degrees, 15 degrees, 20 degrees, 30 degrees, or the like. In some embodiments, a whole or at least a portion of the 2D compass 2402, such as the anatomical direction indicators 2406a-d or the angular index/scale 2404, may be overlaid on the endoscopic view.

The anatomical direction indicators 2406a-d and the angular index/scale 2404 can help an operator understand current orientation of an endoscope capturing an image for the endoscopic view. The anatomical direction indicators 2406a-d of the 2D compass 2402 can inform an operator that “up” will articulate the endoscope toward Anterior, “down” toward Posterior, “left” toward Medial into a bronchus, and “right” toward Lateral into the other bronchus. If the endoscope is “rolled,” in some embodiments, the image may be rotated clockwise or counterclockwise based on an angular change of the “roll” while the anatomical direction indicators 2406a-d and the angular index/scale 2404 remain fixed. In other embodiments, the image may remain fixed while the anatomical direction indicators 2406a-d and the angular index/scale 2404 rotates based on the angular change of the “roll.”

In an example, the 2D compass 2402 is always displayed for an operator over the endoscopic view (e.g., always on) during a procedure. In another example, the compass 2404 can be manually toggled on or off using a switch 2410 by the operator, for example by selecting “OFF” or “2D.” In another example, the compass 2402 or a portion thereof may be automatically toggled on or off depending on the direction of the endoscope. For example, when the endoscope or camera is detected as being exactly or approximately parallel to a specific axis (e.g., parallel to an axis in a cartesian space), the one or more other perpendicular or parallel axes may automatically appear or disappear. In some embodiments, when the endoscope or camera is detected as being exactly or approximately parallel to a specific axis, the 2D compass 2402 may automatically toggle on.

While there are four letters shown on the 2D compass 2402 of FIG. 24, in another example, any other number of letters may be shown. For example, three letters may be shown if an operator has navigated the endoscope to a corner position and there is a dead-end in a certain navigational direction. In another example, a fifth letter may appear/disappear in the middle of the endoscopic view with a softer or deemphasized shading as the operator moves in the direction of the otherwise not displayed parallel axis—for example, in FIG. 24, “M” may appear as the operator moves in the Medial direction and “L” may appear as the operator moves in the Lateral direction.

FIG. 25 illustrates an example of a GUI 2500 including an example of a 3D compass 2502. The 3D compass 2502 may include some or all indicators of a 2D compass (e.g., the 2D compass 2402 of FIG. 24) and their features. The 3D compass 2502 may be provided around the peripheral boundary of or overlaid on an endoscopic view (e.g., the endoscopic view 2202 of FIG. 22) to provide an operator with a better sense of direction as they are navigating an endoscope intraoperatively. For example, the 3D compass 2502 may include 2D anatomical direction indicators (e.g., the anatomical direction indicators 2406a-d of FIG. 24) at different positions along the peripheral boundary of the compass to indicate a corresponding anatomical direction such as Anterior/Posterior, Medial/Lateral. Additionally, the 3D compass 2502 may include 3D anatomical direction indicators 2506a-b at different positions within the endoscopic view to indicate a corresponding anatomical direction such as Superior/Inferior, denoted respectively with letters “S” and “I.” In the illustrated example, the 3D compass is overlaid over the endoscopic view such that the endoscopic view can be viewed through the compass.

The 3D anatomical direction indicators 2506a-b may be positioned on the endoscopic view based on pitch and yaw of a tip of the endoscope. In some embodiments, curvable lines 2504a-b may be presented to indicate pitch and yaw of the endoscope. For example, one or more curvable horizontal lines, which may also be referred as “latitude lines” can be generated connecting each latitude of a 2D axis, such as the M/L axis as shown, to indicate yaw of the endoscope. Similarly, one or more curvable vertical lines, which may also be referred as “longitude lines” can be generated connecting each longitude of another 2D axis, such as the A/P axis as shown, to indicate pitch of the endoscope. It is noted that the curvable lines are “curvable” but may not necessarily be curved in all instances. For example, when the pitch or yaw of the endoscope is aligned exactly with a 2D axis, the curvable horizontal line 2504a or the curvable vertical line 2504b may become a straight line.

For simplicity, the example 3D compass 2502 as shown is illustrated to show only a curvable horizontal line 2504a and a curvable vertical line 2504b. Each of the curvable lines 2504a-b can envelope the endoscopic view as if the endoscopic view is a sphere. In some embodiments, each of the curvable lines 2504a-b may indicate proximal portions of the sphere and distal portions of the sphere by differentiating various aspects of the curvable lines 2504a-b. As an example, the curvable lines 2504a-b are illustrated solid for the proximal portions and broken for the distal portions. In some other implementations, other differentiating characteristics such as line thickness (e.g., thicker for the proximal portions compared to the distal portions), line color, line contrast, line softness, line presence (e.g., only one of the proximal portions or the distal portions presented), or the like may be used. In an example, the letter or letters displayed in the actual field-of-view farther from the user may be deemphasized or provided with less brightness or contrast.

The 3D anatomical direction indicators 2506a-b may be positioned at intersections of the curvable lines 2504a-b. As shown, the curvable horizontal line 2504a and the curvable vertical line 2504b intersects at two points, at a proximal intersection on the proximal portion of the sphere and at a distal intersection on the distal portion of the sphere. An axis that connects the two points can be the Superior/Inferior axis for the 3D compass 2502. Accordingly, an anatomical direction indicator (e.g., “S”) can be positioned at the proximal intersection and another anatomical direction indicator (e.g., “I”) can be positioned at the distal intersection.

It should be appreciated that while anatomical indicators (S/I, A/P, M/L) are used in FIGS. 24-25, other symbols or indicators may be used to inform the user of the anatomical directions and, in some instances, other anatomical indicators along other axes including Proximal/Distal, Central/Peripheral, Superficial/Deep, Dorsal/Ventral, or the like may be used.

Referring back to both FIGS. 24-25, in addition to the anatomical direction indicators (S/I, A/P, M/L) 2406a-d, 2506a-b on the boundary of the 2D compass 2402 and 3D compass 2502, respectively, a target site indicator 2408 in FIG. 24 showing location of a target site in the endoscopic view may also be displayed on the compasses 2402, 2502. This target indicator 2408 may be an icon, a shape, a mask, a symbol, a letter, or some other indicator on the peripheral boundary of the compasses 2402, 2502 or otherwise displayed within an actual field-of-view of the endoscopic view. In some embodiments, the target site indicator 2408 may become larger, darker, brighter, change color or contrast, or the like based on a distance of the endoscope to the target site. The target site indicator 2408 can assist the operator with identifying the direction in which they should advance the endoscope in order to reach a target anatomical site (e.g., a nodule or a legion).

In some embodiments, one or more additional visual indicators may be presented to indicate to the operator whether they are properly center-driving the endoscope. As non-limiting examples, the visual indicators can be a broken or solid line (not shown), an icon, a shape, a symbol, a letter, or other graphical elements. It can be advantageous for navigation of the endoscope to be central with respect to the airway or anatomical channel where the tool or endoscope is being driven, this is referred to as center-driving. An indicator such as a bullseye may indicate to the user how best to drive the endoscope or tool in order to achieve center-driving.

Still referring to FIGS. 24-25, a compass is designed to give a user a better sense of direction as they are navigating and adjusting scope tip position during a procedure. For example, as users navigate in the periphery of the lungs, they may lose their sense of direction and face a large cognitive burden to determine the anatomic direction that the scope tip is pointing. This is particularly important for customers who use advanced imaging and need to know how to adjust their scope position based on the imaging results. The purpose of the compass is to reduce the cognitive load on the user to allow them to easily recognize what anatomic direction up/down/left/right/insertion/retraction correspond to on the controller. This is achieved by providing the user corresponding anatomical directions (Superior/Inferior, Anterior/Posterior, Medial/Lateral) in the live endoscopic view.

In some embodiments, an orientation indicator (e.g., the orientation indicator 2310, 2320, 2330 of FIG. 22) or a compass (e.g., the compasses 2402, 2502 of FIGS. 24-25) may be always presented to the user. In other embodiments, the orientation indicator or the compass can be manually toggled on or off using a selectable UI element (e.g., a toggle button 2302 of FIG. 23 or the switch 2410 of FIG. 24). The selectable UI element may also be used to transition a GUI from presenting an overlayed compass as shown in FIGS. 24-25 to an adjacent orientation indicator 2310 as shown in FIG. 23, or vice versa.

FIG. 26 illustrates example articulation scenarios 2600 in association with articulation indicators 2604, 2606. The articulation indicators 2600 may be presented adjacent to a peripheral boundary of (e.g., the articulation indicator 2340 of FIG. 23), surrounding the peripheral boundary as shown in the example articulation scenarios 2600(a)-(e), or overlaid on an endoscopic view (e.g., the endoscopic view 2202 of FIG. 22). The articulation indicators 2600 can inform an operator of various articulation states including: at least one selected articulable component, current articulation of the articulable component, maximum available articulation of the articulable component, articulability of the articulable component, or the like.

The articulation indicator can include a component selector 2602 that can select one or more articulable components of an endoscope for articulation. In a first scenario 2600(a), the component selector 2602 has selected “PAIRED” (both the sheath portion and the leader portion) for articulation. In a second scenario 2600(b), the component selector 2602 has selected “SHEATH” (the sheath portion only) for articulation. In a third scenario 2600(c), a fourth scenario 2600(d), and a fifth scenario 2600(e), the component selector 2602 has selected “SCOPE” (the leader portion only) for articulation.

Each of the scenarios 2600(a)-(e) can have a sheath articulation indicator 2604 and a scope articulation indicator 2606. The shown example scenarios 2600(a)-(e) present the sheath articulation indicator 2604 as surrounding the scope articulation indicator 2606, which may be more instinctive, but it may be reversed in some implementations. As shown in the scenarios 2600(a)-(e), one or more selected articulation indicators can be emphasized, such as with greater contrast, highlight, gradient, pattern, color, or the like, compared to one or more unselected articulation indicators. For example, in the first scenario 2600(a), both the sheath articulation indicator 2604 and the scope articulation indicator 2606 are emphasized. As another example, in the second scenario 2600(b), only the sheath articulation indicator 2604 is emphasized. As yet another example, in the third, fourth, and fifth scenarios 2600(c)-(e), only the scope articulation indicator 2606 is emphasized.

The articulation indicators 2604, 2606 can be split into multiple sections (e.g., quadrants as shown) and be positioned in about the peripheral boundary of a compass (e.g., the 2D compass 2402 of FIG. 23 or the 3D compass 2502 of FIG. 25). In some implementations, the articulation indicators 2604, 2606 can be split into more or fewer sections such as into five, six, eight, twelve, or any number of sections. Each section can represent articulation toward a direction enveloped by the section. Further, each section can represent a magnitude of articulation (e.g., a degree of articulation). Accordingly, the sections can represent an articulation vector having a direction and magnitude to an operator.

Continuing with the example scenarios 2600(a)-(e) split into quadrants, each section can be represented with a corresponding arc. It is noted that in the example scenarios 2600(a)-(e), each arc is aligned with an anatomical direction (e.g., A/P, M/L) but the alignment is for ease of description. Positioning of the arcs may depend on endoscope control scheme whereas positioning of the anatomical directions may depend on one or more reference axes determined with the calibration procedure described in FIG. 23. In other words, the positioning of the arcs may be based on control characteristics of the endoscope and the positioning of the anatomical directions may be based on placement of a patient or a luminal network within the patient. In some embodiments, when the endoscope is “rolled,” the arcs may be rolled together with a corresponding amount. In some other embodiments, the arcs may remain fixed during the endoscope roll.

Lengths of an arc can indicate an maximum available articulation of the endoscope toward the arc. When the endoscope is articulated toward the arc, a magnitude of articulation may be represented, as shown, by filling the arc until the maximum available articulation is reached. For example, in the first scenario 2600(a), both the sheath articulation indicator 2604 and the scope articulation indicator 2606 indicate articulation toward Anterior and Lateral as illustrated with approximately half-filled articulation indicators 2604a, 2604a′, 2606a, 2606a′. However, the endoscope is not articulated toward Posterior as illustrated with empty articulation indicators 2604a″, 2606a″. Similarly, the endoscope is not articulated toward Medial. The approximately half-filled articulation indicators 2604a, 2604a′, 2606a, 2606a′ indicate that the endoscope is articulated to approximately half the maximum available articulation toward Anterior and Lateral. Thus, the articulation indicators 2604, 2606 can inform an operator of current articulation vectors of the sheath portion and the leader portion.

In the second scenario 2600(b), only the sheath portion is selected for articulation as indicated by the emphasized sheath articulation indicator 2604b in contrast with the de-emphasized scope articulation indicator 2606b. The second scenario 2600(b) has the same articulation vectors as the first scenario 2600(a). If the endoscope were to be articulated further toward Anterior based on the selection, the sheath articulation indicator 2604b will further fill whereas the scope articulation indicator 2606b remains unchanged.

In the third scenario 2600(c), only the leader portion is selected for articulation as indicated by the emphasized leader articulation indicator 2606c in contrast with the de-emphasized sheath articulation indicator 2604c. The third scenario 2600(c) has a first articulation vector of the sheath portion toward Posterior and Medial and a second articulation vector of the leader portion toward Anterior and Lateral. If the endoscope were to be articulated further toward Anterior based on the selection, the scope articulation indicator 2606c will further fill whereas the sheath articulation indicator 2604c remains unchanged.

In the fourth scenario 2600(d), a first completely filled scope articulation indicator 2606d may indicate maximum articulation and, thus, the leader portion may not be articulated further toward Anterior. Similarly, a second completely filled scope articulation indicator 2606d′ may indicate maximum articulation and, thus, the leader portion may not be articulated further toward Lateral. In some embodiments, when the maximum articulation has been reached, one or more properties of the articulation indicator (e.g., color, thickness, pattern, or the like) can be changed to indicate that an articulable component is at its maximum articulation.

In the fifth scenario 2600(e), the leader portion has not yet reached its maximum articulation toward Anterior as the scope articulation indicator 2606e is not completely filled. However, here, the leader portion may not continue articulation toward Anterior because such articulation may be obstructed by a wall or an object in the Anterior anatomical direction. In such scenario, attempting to articulate the leader portion further toward Anterior may apply too much force and thereby cause the endoscope to buckle or a pull wire to snap under stress. The undesirable stress can be indicated by the scope articulation indicator 2606e showing emphasis (e.g., changing property of the articulation indicator 2606e) near the outer edges. For example, the articulation indicator 2606e is shown as having changed its color near its outer edges.

Although FIG. 26 and its scenarios 2600(a)-(e) illustrate arc-based articulation indicators 2604, 2606 to represent articulation vectors, it is noted that other implementations are possible. For example, the articulation indicator 2340 of FIG. 23 or a vector centered at an endoscopic view may be contemplated.

Additionally, thus far, orientation indicators (e.g., the orientation indicator 2310 of FIG. 23 and compasses 2402, 2502 of FIGS. 24-25) have been described as applicable to live endoscopic views. However, it is also contemplated that the orientation indicators and various features thereof may be applied to a previously captured endoscopic image. For example, the present disclosure may be applied to a previously captured and stored sequence of endoscopic images to add the orientation indicators to the endoscopic images post-capture.

### 3. Implementing Systems and Terminology

Implementations disclosed herein provide systems, methods and apparatus for user interfaces for navigating anatomical channels in medical procedures.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. It should be noted that a computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

The previous description of the disclosed implementations is provided to enable any person skilled in the art to make or use the present invention. Various modifications to these implementations will be readily apparent to those skilled in the art, and the generic principles defined herein may be applied to other implementations without departing from the scope of the invention. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present invention is not intended to be limited to the implementations shown herein but is to be accorded the widest scope consistent with the principles and novel features disclosed herein.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A medical system, comprising:
an instrument configured to be inserted into an anatomy;
a camera disposed on a distal end of the instrument and configured to capture images of the anatomy within its field-of-view (FOV); and
a control system configured to:
display the images in a graphical user interface (GUI);
determine an orientation of the FOV relative to the anatomy; and
generate a compass for display with the images on the GUI, the compass indicating the orientation of the FOV depicted by the images relative to the anatomy.

2. The medical system of claim 1, wherein the control system is further configured to:
receive user input for toggling the compass on or off; and
selectively display the compass on the GUI based at least in part on the received user input.

3. The medical system of claim 1, wherein the control system is further configured to selectively display the compass on the GUI based at least in part on a position, orientation, or movement direction of the instrument.

4. The medical system of claim 1, wherein the control system is further configured to:
determine whether a movement of the instrument is parallel to a first axis in a cartesian space; and
display the compass on the GUI responsive to determining that the movement of the instrument is parallel to the first axis, the compass indicating one or more axes of the cartesian space that are perpendicular to the first axis.

5. The medical system of claim 1, wherein the control system is further configured to change an appearance of one or more direction indicators associated with the compass based at least in part on a position, orientation, or movement direction of the instrument.

6. The medical system of claim 1, wherein the GUI further comprises a target site indicator indicating a distance or direction of a target relative to the orientation of the FOV.

7. The medical system of claim 1, wherein the GUI further comprises an indicator indicating a center of an anatomical channel within which the camera is disposed relative to the orientation of the FOV.

8. The medical system of claim 1, wherein the compass includes an orientation indicator indicating at least a first anatomical direction and a second anatomical direction associated with a reference axis of the anatomy.

9. The medical system of claim 8, wherein the orientation indicator is a two-dimensional (2D) orientation indicator having a first axis associated with anterior and posterior anatomical directions and having a second axis associated with medial and lateral anatomical directions.

10. The medical system of claim 8, wherein the orientation indicator is a three-dimensional (3D) orientation indicator having a first axis associated with anterior and posterior anatomical directions, a second axis associated with medial and lateral anatomical directions, and a third axis associated with superior and inferior anatomical directions based on yaw and pitch of the instrument.

11. The medical system of claim 1, wherein the control system is further configured to:
determine a degree of articulation by the instrument;
determine a direction of articulation by the instrument; and
display an articulation indicator on the GUI having a length indicating the degree of articulation and a position relative to the GUI indicating the direction of articulation.

12. The medical system of claim 11, wherein the control system is further configured to:
determine that the degree of articulation by the instrument is equal to a maximum articulation; and
change at least one property associated with the articulation indicator based on determining that the degree of articulation by the instrument is equal to the maximum articulation.

13. The medical system of claim 11, wherein the control system is further configured to:
determine that the instrument is obstructed from further articulation; and
change at least one property associated with the articulation indicator based on determining that the instrument is obstructed from further articulation.

14. The medical system of claim 1, wherein the control system is further configured to:
configure the images to depict an expanded view of the FOV or a restricted view of the FOV for display in the GUI, the expanded view representing the FOV in its entirety and the restricted view representing only a portion of the FOV.

15. The medical system of claim 14, wherein the control system is further configured to toggle between the expanded view and the restricted view for display on the GUI responsive to user input.

16. The medical system of claim 14, wherein the control system is further configured to toggle between the restricted view and the expanded view in response to detecting one or more conditions associated with navigating the instrument within the anatomy.

17. The medical system of claim 14, wherein the expanded view includes one or more simulated image portions.

18. A method performed by a medical system, comprising:
receiving images of an anatomy captured within a field-of-view (FOV) of a camera disposed on a distal end of an instrument configured to be inserted into the anatomy;
displaying the images in a graphical user interface (GUI);
determining an orientation of the FOV relative to the anatomy; and
generating a compass for display with the images on the GUI, the compass indicating the orientation of the FOV depicted by the images relative to the anatomy.

19. The method of claim 18, further comprising:
determining a degree of articulation by the instrument;
determining a direction of articulation by the instrument; and
displaying an articulation indicator on the GUI having a length indicating the degree of articulation and a position relative to the GUI indicating the direction of articulation.

20. The method of claim 18, further comprising:
configuring the images to depict an expanded view of the FOV or a restricted view of the FOV for display in the GUI in response to user input or detecting one or more conditions associated with navigating the instrument within the anatomy, the expanded view representing the FOV in its entirety and the restricted view representing only a portion of the FOV.
